(19) United States
(12) Patent Application Publication
(10) Pub. No.: US 2015/0289896 A1
(43) Pub. Date: Oct. 15, 2015

(54) FLUID EJECTION DEVICE

(71) Applicant: SEIKO EPSON CORPORATION, Tokyo (JP)

(72) Inventors: Masaki GOMI, Hino-shi (JP); Kazuaki UCHIDA, Fujimi-machi (JP)

(21) Appl. No.: 14/682,939

(22) Filed: Apr. 9, 2015

(30) Foreign Application Priority Data
Apr. 10, 2014 (JP) 2014-080831

(51) Int. Cl. A61B 17/3203; A61B 17/3209; A61B 17/3205

(52) U.S. Cl. CPC A61B 17/3203; A61B 17/3205; A61B 17/3209

(57) ABSTRACT

A fluid ejection device includes a fluid accommodation portion that has a fluid outlet connected to a fluid connection tube having a channel. A fluid pressing unit causes the fluid to flow out of the fluid outlet. A fluid ejection unit ejects in a pulsed manner fluid received from the fluid connection tube. A pressure detection unit detects pressure when the fluid pressing unit operates. A control unit causes the fluid pressing unit to operate in a state in which the channel is closed, and determines that the pressure detection unit has failed if the detected pressure when the fluid pressing unit comes into contact with the fluid accommodation portion is lower than a first determination value, or if the detected pressure when the fluid pressing unit does not come into contact with the fluid accommodation portion is equal to or higher than a second determination value.

This application claims the benefit of Japanese Patent Application No. 2014-080831, filed on Apr. 10, 2014. The content of the aforementioned application is incorporated herein by reference in its entirety.

### BACKGROUND

1. Technical Field

The present invention relates to a fluid ejection device.

2. Related Art

A technology in which an object is incised or excised by ejecting a pulsed fluid is known. For example, in the medical field, a fluid ejection device is proposed as an operation scalpel to incise or excise living tissue, the fluid ejection device being configured to include a pulsation generator that ejects a pulsed fluid, a fluid supply unit that supplies a fluid to the pulsation generator, and a fluid supply path that connects the fluid supply unit to the pulsation generator (refer to JP-A-2013-213422).

In the fluid ejection device, it is important to maintain the fluid in a fluid supply path at an appropriate pressure and to cause the fluid to be ejected from the pulsation generator at an appropriate intensity. For this reason, the fluid ejection device has a pressure sensor for detecting the inner fluid pressure of the fluid supply path; however, the pressure sensor may fail.

If the pressure sensor fails, the inner fluid pressure of the fluid supply path cannot be controlled to be an appropriate pressure, and thus there is a possibility that the fluid may be ejected from the pulsation generation unit at an unintended intensity.

### SUMMARY

An advantage of some aspects of the invention is to provide a technology for realizing a fluid ejection device which detects a failure in such a pressure sensor and has higher safety and reliability.

A fluid ejection device according to an aspect of the invention includes: a fluid container that has a fluid accommodation portion for accommodating a fluid, and a fluid outlet formed in the fluid accommodation portion; a fluid pressing unit that presses the fluid accommodation portion to cause the fluid to flow out of the fluid outlet; a connection tube, one end of which is connected to the fluid outlet; a fluid ejection unit that has a fluid intake port connected to the other end of the connection tube, and ejects in a pulsed manner the fluid taken in via the fluid intake port; a channel opening and closing unit that opens and closes a channel of the fluid in the connection tube; a pressure detection unit that detects a pressure when the fluid pressing unit presses the fluid accommodation portion and outputs a detection signal with a level corresponding to the pressure; and a control unit that causes the fluid pressing unit to press the fluid accommodation portion in a state in which the fluid opening and closing unit closes the channel, and determines that the pressure detection unit has failed in a case where the pressure indicated by the detection signal when the fluid pressing unit comes into contact with the fluid accommodation portion is lower than a first determination value, or in a case where the pressure indicated by the detection signal when the fluid pressing unit does not come into contact with the fluid accommodation portion is equal to or higher than a second determination value.

Other features of the invention will be made apparent by the description of this specification and the accompanying drawings.

### BRIEF DESCRIPTION OF THE DRAWINGS

The invention will be described with reference to the accompanying drawings, wherein like numbers reference like elements.

FIG. 1 is a block diagram illustrating an example of the entire configuration of a fluid ejection device according to an embodiment of the invention.

FIG. 2 is a block diagram illustrating another example of the entire configuration of the fluid ejection device according to the embodiment of the invention.

FIG. 3 is a block diagram illustrating the configuration of a pump according to the embodiment of the invention.

FIG. 4 is a block diagram illustrating the configuration of the pump according to the embodiment of the invention.

FIG. 5 is a cross-sectional view illustrating the structure of a pulsation generator according to the embodiment of the invention.

FIG. 6 is a plan view illustrating the shape of an inlet channel according to the embodiment of the invention.

FIG. 7 is a block diagram illustrating the configuration of a pump control unit according to the embodiment of the invention.

FIG. 8 is a diagram illustrating a level of a detection signal of a pressure sensor and a transition in an output signal of a touch sensor according to the embodiment of the invention.

FIG. 9 is a flowchart illustrating a flow of a process of the pump control unit according to the embodiment of the invention.

FIG. 10 is a diagram illustrating a level of a detection signal of a pressure sensor and a transition in an output signal of a touch sensor according to the embodiment of the invention.

FIG. 11 is a flowchart illustrating a flow of a process of the pump control unit according to the embodiment of the invention.

FIG. 12 is a diagram illustrating a configuration example of a slider according to the embodiment of the invention.

### DESCRIPTION OF EXEMPLARY EMBODIMENTS

### Outline

At least the following facts are apparent from this specification and the accompanying drawings.

A fluid ejection device includes: a fluid container that has a fluid accommodation portion for accommodating a fluid, and a fluid outlet formed in the fluid accommodation portion; a fluid pressing unit that presses the fluid accommodation portion to cause the fluid to flow out of the fluid outlet; a connection tube, one end of which is connected to the fluid outlet; a fluid ejection unit that has a fluid intake port connected to the other end of the connection tube, and ejects in a pulsed manner the fluid taken in via the fluid intake port; a channel opening and closing unit that opens and closes a channel of the fluid in the connection tube; a pressure detection unit that detects a pressure when the fluid pressing unit presses the fluid accommodation portion and outputs a detection signal with a level corresponding to the pressure; and a control unit that causes the fluid pressing unit to press the fluid accommodation portion in a state in which the fluid opening and closing unit closes the channel, and determines that the pressure detection unit has failed in a case where the pressure indicated by the detection signal when the fluid pressing unit comes into contact with the fluid accommodation portion is lower than a first determination value, or in a case where the pressure indicated by the detection signal when the fluid pressing unit does not come into contact with the fluid accommodation portion is equal to or higher than a second determination value.

It is possible to implement the fluid ejection device which detects a failure in the pressure detection unit and has higher safety and reliability.

In the fluid ejection device, it is preferable that the fluid pressing unit includes a moving body that moves in a pressing direction of the fluid accommodation portion so as to press the fluid accommodation portion; and a position detection unit that detects a position of the moving body, and the first determination value is a value which is set on the basis of the pressure indicated by the detection signal output from the pressure detection unit when the moving body is located at a predetermined position where the moving body does not come into contact with the fluid accommodation portion.

The fluid ejection device can detect a failure in the pressure detection unit even if a pressure used as a reference when detecting a failure in the pressure detection unit is deviated from a regular pressure.

In the fluid ejection device, it is preferable that the first determination value is a value obtained by adding a predetermined value defined according to an error of the pressure to the pressure indicated by the detection signal output from the pressure detection unit when the moving body is located at the predetermined position.

The fluid ejection device can remove the influences of errors of the pressure detection unit when detecting a failure in the pressure detection unit.

It is preferable that the fluid ejection device further includes a first pressure detection unit and a second pressure detection unit, and the first pressure detection unit and the second pressure detection unit are disposed so as to be stacked in the pressing direction of the moving body at a position where the pressure detection units are clamped by the moving body and the fluid accommodation unit when the moving body presses the fluid accommodation portion, and the control unit controls the movement of the moving body so that a pressure when the moving body presses the fluid accommodation portion becomes a predetermined target pressure value by using either one or both of a first detection signal output from the first pressure detection unit and a second detection signal output from the second pressure detection unit, continuously performs the control by using the second detection signal in a case where it is determined that the first pressure detection unit has failed, and continuously performs the control by using the first detection signal in a case where it is determined that the second pressure detection unit has failed.

The fluid ejection device can continuously perform the control of ejection of the fluid from the fluid ejection unit even if the first pressure detection unit or the second pressure detection unit has failed, and thus it is possible to improve the reliability of the fluid ejection device.

In the fluid ejection device, it is preferable that the control unit causes the fluid opening and closing unit to open the channel in a case where it is determined that the pressure detection unit has failed.

In the fluid ejection device, even if an inner pressure of the fluid accommodation portion is higher than an expected pressure, the fluid in the fluid accommodation portion is made to flow out of the fluid ejection unit so that the inner pressure of the fluid accommodation portion can be reduced, and thus it is possible to improve the safety of the fluid ejection device.

In the fluid ejection device, it is preferable that the control unit outputs an alarm indicating that the pressure detection unit has failed in a case where it is determined that the pressure detection unit has failed.

The fluid ejection device can promptly notify an operator such as a practitioner that the pressure detection unit has failed, and thus it is possible to further improve the safety of the fluid ejection device.

### Entire Configuration

Hereinafter, an embodiment of the invention will be described with reference to the accompanying drawings. A fluid ejection device according to the embodiment can be used in various procedures such as the cleaning or cutting of a fine object or structure, living tissue, or the like; however, an example of the embodiment given in the following description is the fluid ejection device suitable for use as an operation scalpel to incise or excise living tissue. Accordingly, a fluid used in the fluid ejecting device according to the embodiment is water, physiologic saline, a predetermined fluid medicine, or the like. The drawings referenced in the following description are schematic views in which a portion not being as defined a member is vertically and horizontally scaled differently from an actual scale for illustrative purposes.

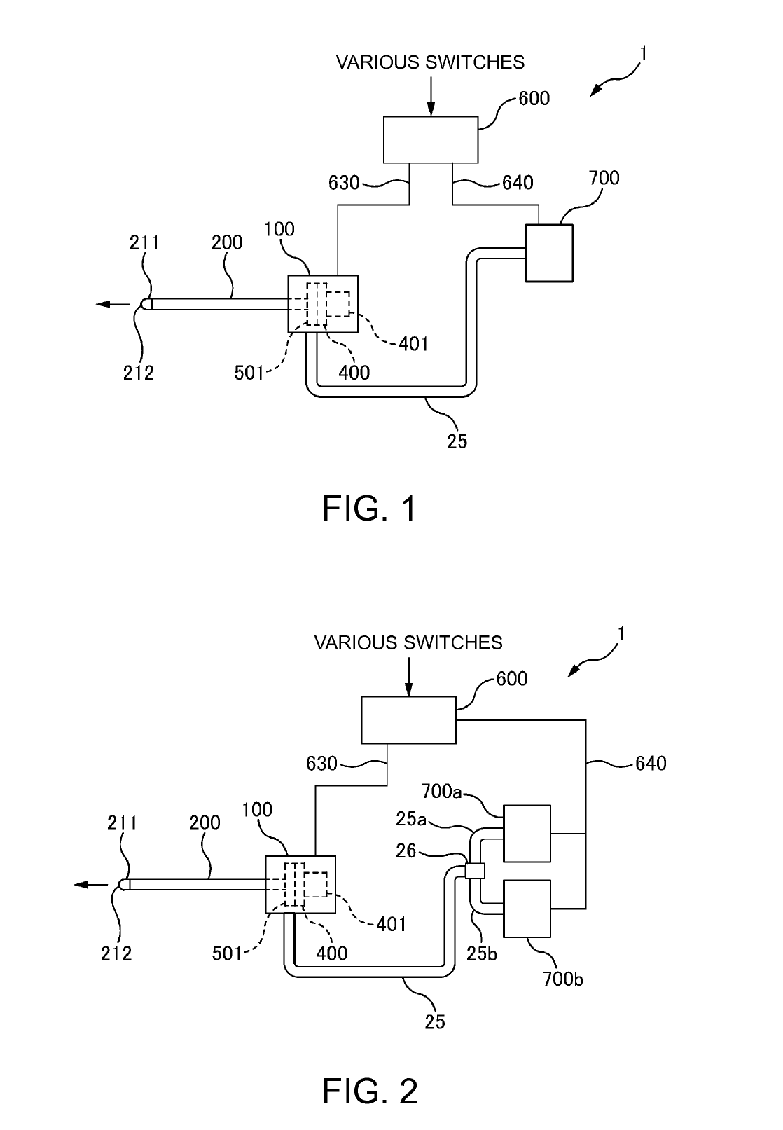

FIG. 1 is a view illustrating the configuration of a fluid ejection device 1 as an operation scalpel according to the embodiment. The fluid ejection device 1 according to the embodiment includes a pump 700 for supplying a fluid; a pulsation generator (a fluid ejection unit) 100 that converts a flow of the fluid supplied from the pump 700 into a pulsed flow, and ejects the fluid in a pulsed manner; a drive control unit 600 that controls the fluid ejection device 1 in cooperation with the pump 700; and a connection tube (connection path) 25 acting as a channel through which the pump 700 and the pulsation generator 100 is connected to each other, and the fluid flows.

The pulsation generator 100 includes a fluid chamber 501 that accommodates the fluid supplied from the pump 700; a diaphragm 400 that changes the volume of the fluid chamber 501; and a piezoelectric element 401 that vibrates the diaphragm. 400, all of which will be described later in detail.

The pulsation generator 100 includes a thin pipe-like fluid ejection tube 200 that acts as a channel of the fluid discharged from the fluid chamber 501, and a nozzle 211 that is mounted on a tip end portion of the fluid ejection tube 200 and has a reduced channel diameter.

The pulsation generator 100 converts a flow of the fluid into a pulsed flow by applying a pulsed pressure to the fluid via the driving of the piezoelectric element 401 in response to drive signals output from the drive control unit 600 and the changing of the volume of the fluid chamber 501, and the pulsation generator 100 ejects the fluid in a pulsed manner via the fluid ejection tube 200 and the nozzle 211.

The drive control unit 600 and the pulsation generator 100 are connected to each other via a control cable 630, and drive signals for driving the piezoelectric element 401 are output from the drive control unit 600, and are transmitted to the pulsation generator 100 via the control cable 630.

The drive control unit 600 and the pump 700 are connected to each other via a communication cable 640, and the drive control unit 600 and the pump 700 transmit and receive various commands or data therebetween according to a predetermined communication protocol such as a controller area network (CAN).

The drive control unit 600 receives signals from various switches operated by a practitioner who performs an operation using the pulsation generator 100, and controls the pump 700 or the pulsation generator 100 via the control cable 630 or the communication cable 640.

The switches that input signals to the drive control unit 600 are a pulsation generator start-up switch 625, an ejection intensity switching switch 627, a flushing switch 628, and the like (not illustrated).

The pulsation generator start-up switch 625 is a switch for switching between the ejection and the non-ejection of the fluid from the fluid ejection unit 100. When a practitioner who performs an operation using the pulsation generator 100 operates the pulsation generator start-up switch 625, the drive control unit 600 controls the pulsation generator 100 to eject the fluid or stop the ejection of the fluid in cooperation with the pump 700. The pulsation generator start-up switch 625 can be a switch configured to be operated by the practitioner's feet, or a switch that is provided integrally with the pulsation generator 100 grasped by the practitioner, and configured to be operated by the practitioner's hands or fingers.

The ejection intensity switching switch 627 is a switch for changing the intensity of fluid ejection from the pulsation generator 100. When the ejection intensity switching switch 627 is operated, the drive control unit 600 controls the pulsation generator 100 and the pump 700 so as to increase and decrease the intensity of fluid ejection.

The flushing switch 628 will be described later.

In the embodiment, a pulsed flow implies a flow of a fluid, a flow direction of which is constant, and the flow rate or flow speed of which is changed periodically or non-periodically. The pulsed flow may be an intermittent flow in which the flowing and stopping of the fluid are repeated; however, since the flow rate or flow speed of the fluid is preferably changed periodically or non-periodically, the pulsed flow is not necessarily an intermittent flow.

Similarly, the ejection of a fluid in a pulsed form implies the ejection of the fluid by which the flow rate or moving speed of an ejected fluid is changed periodically or non-periodically. An example of the pulsed ejection is an intermittent ejection by which the ejection and non-ejection of a fluid are repeated; however, since the flow rate or moving speed of an ejected fluid is preferably changed periodically or non-periodically, the pulsed ejection is not necessarily an intermittent ejection.

When the driving of the pulsation generator 100 is stopped, that is, when the volume of the fluid chamber 501 is not changed, the fluid supplied from the pump 700 as a fluid supply unit at a predetermined pressure continuously flows out of the nozzle 211 via the fluid chamber 501.

The fluid ejection device 1 according to the embodiment maybe configured to include a plurality of the pumps 700. FIG. 2 illustrates an example of the configuration of the fluid ejection device 1 configured to include two pumps 700.

In this case, as illustrated in FIG. 2, the fluid ejection device 1 includes a first pump 700a and a second pump 700b. A first connection tube 25a, a second connection tube 25b, the connection tube 25, and a three way stopcock 26 form a connection path (connection tube) which connects the pulsation generator 100 and the first pump 700a, and the pulsation generator 100 and the second pump 700b, and acts as a channel through which the fluid flows.

The three way stopcock 26 is a valve configured to be able to communicate the first connection tube 25a and the connection tube 25, or the second connection tube 25b and the connection tube 25, and either one of the first pump 700a and the second pump 700b is selectively used.

In this configuration, for example, when the first pump 700a cannot supply the fluid for unknown reasons such as a malfunction while being selected and used, it is possible to continuously use the fluid ejection device 1 and to minimize adverse effects associated with the non-supply of the fluid from the first pump 700a by switching the three way stopcock 26 so as to communicate the second connection tube 25b and the connection tube 25, and starting the supply of the fluid from the second pump 700b.

When the fluid ejection device 1 is configured to include a plurality of the pumps 700, but the pumps 700 are not required to be distinctively described, in the following description, the pumps 700 are collectively expressed by the pump 700.

In contrast, when the plurality of pumps 700 are required to be distinctively described, suffixes such as “a” and “b” are properly added to reference sign 700 of the pump, and each of the pumps 700 is distinctively expressed by the first pump 700a or the second pump 700b. In this case, each configuration element of the first pump 700a is expressed by adding the suffix “a” to a reference sign of each configuration element, and each configuration element of the second pump 700b is expressed by adding the suffix “b” to a reference sign of each configuration element.

### Pump

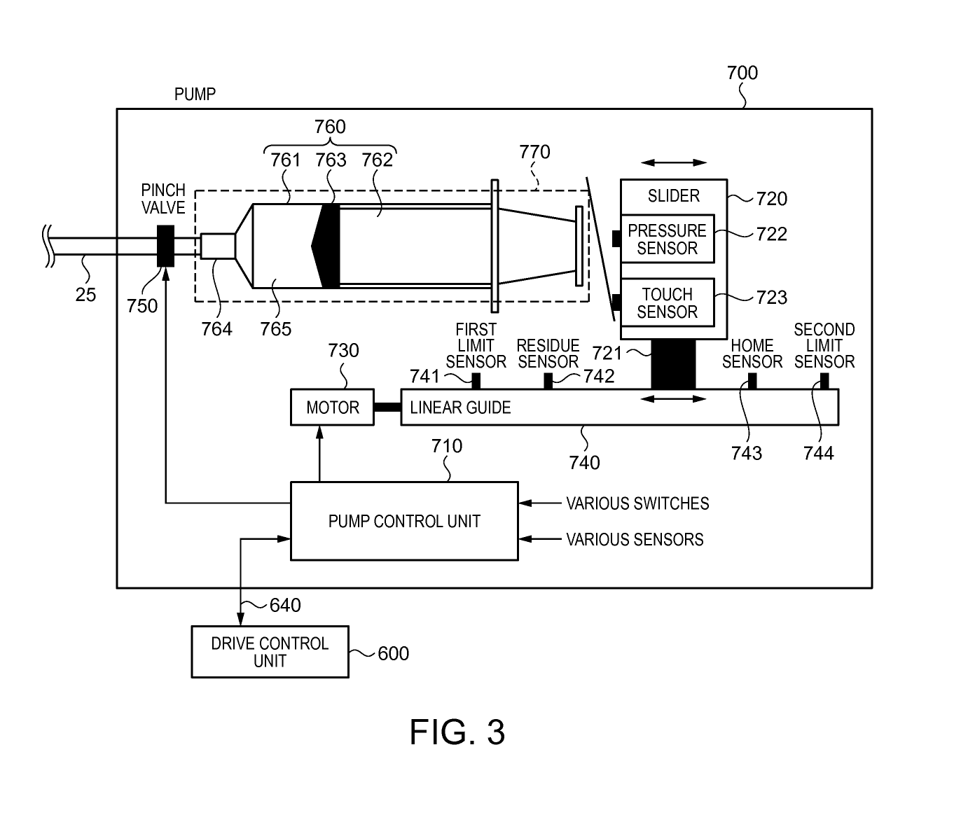

Subsequently, an outline of the configuration and operation of the pump 700 according to the embodiment will be described with reference to FIG. 3.

The pump 700 according to the embodiment includes a pump control unit (a control unit) 710; a slider (moving body) 720; a motor 730; a linear guide 740; and a pinch valve (a channel opening and closing unit) 750. The pump 700 is configured to have a fluid container mounting unit 770 for attachably and detachably mounting a fluid container 760 that accommodates the fluid. The fluid container mounting unit 770 is formed so as to hold the fluid container 760 at a specific position when the fluid container 760 is mounted thereon.

The following switches (which will be described later in detail) (not illustrated) input signals to the pump control unit 710: a slider release switch 780; a slider set switch 781; a fluid supply ready switch 782; a priming switch 783; and a pinch valve switch 785.

In the embodiment, for example, the fluid container 760 is formed of a medical syringe configured to include a syringe 761 and a plunger 762.

In the fluid container 760, a protrusive cylinder-shaped opening (a fluid outlet) 764 is formed in a tip end portion of the syringe 761. When the fluid container 760 is mounted on the fluid container mounting unit 770, an end portion of the connection tube 25 is inserted into the opening 764, and a fluid channel is formed from the inside of the syringe 761 to the connection tube 25.

The pinch valve 750 is a valve that is provided on a path of the connection tube 25 and opens and closes a fluid channel between the fluid container 760 and the pulsation generator 100.

The pump control unit 710 controls the opening and closing of the pinch valve 750. When the pump control unit 710 opens the pinch valve 750, the fluid container 760 and the pulsation generator 100 communicate with each other via the channel therebetween. When the pump control unit 710 closes the pinch valve 750, the channel between the fluid container 760 and the pulsation generator 100 is shut off.

In a state where the fluid container 760 is mounted on the fluid container mounting unit 770, and the pinch valve 750 is opened, when the plunger 762 of the fluid container 760 moves in a direction (hereinafter, also referred to as a push-in direction or a pressing direction) in which the plunger 762 is pushed into the syringe 761, the volume of a space (hereinafter, also referred to as a fluid accommodation portion 765) is reduced, the space being enveloped by an end surface of a gasket 763 made of resin such as elastic rubber and mounted at the tip of the plunger 762 in the push-in direction, and an inner wall of the syringe 761, and the fluid in the fluid accommodation portion 765 is discharged via the opening 764 of the tip end portion of the syringe 761. The connection tube 25 is filled with the fluid discharged via the opening 764, and the discharged fluid is supplied to the pulsation generator 100.

In contrast, in a state where the fluid container 760 is mounted on the fluid container mounting unit 770, and the pinch valve 750 is closed, when the plunger 762 of the fluid container 760 moves in the push-in direction, it is possible to reduce the volume of the fluid accommodation portion 765, the fluid accommodation portion 765 being enveloped by the gasket 763 mounted at the tip of the plunger 762 and the inner wall of the syringe 761, and it is possible to increase the pressure of the fluid in the fluid accommodation portion 765.

The pump control unit 710 moves the slider 720 along a direction (in the push-in direction and the opposite direction of the push-in direction) in which the plunger 762 moves in a state where the fluid container 760 is mounted on the fluid container mounting unit 770, and the plunger 762 moves in accordance with the movement of the slider 720.

Specifically, the slider 720 is attached to the linear guide 740 in such a manner that a pedestal 721 of the slider 720 engages with a rail (not illustrated) formed linearly on the linear guide 740 along the slide direction of the plunger 762. The linear guide 740 moves the pedestal 721 of the slider 720 along the rail using power transmitted from the motor 730 driven by the pump control unit 710, and thereby the slider 720 moves along the slide direction of the plunger 762.

As illustrated in FIG. 3, the following sensors are provided along the rail of the linear guide 740: a first limit sensor 741; a residue sensor 742; a home sensor (a position detection unit) 743; and a second limit sensor 744.

All of the first limit sensor 741, the residue sensor 742, the home sensor 743, and the second limit sensor 744 are sensors for detecting the position of the slider 720 that moves on the rail of the linear guide 740, and signals detected by these sensors are input to the pump control unit 710.

The home sensor 743 is a sensor used to determine an initial position (hereinafter, also referred to as a home position) of the slider 720 on the linear guide 740. The home position is a position in which the slider 720 is held when the fluid container 760 is mounted or replaced. The home position is set to a position where the slider 720 does not come into contact with the plunger 762 when the slider 720 is located at the home position.

The residue sensor 742 is a sensor for detecting the position (hereinafter, also referred to as a residual position) of the slider 720 when the residue of the fluid in the fluid container 760 becomes less than or equal to a predetermined value while the slider 720 moves from the home position in the push-in direction of the plunger 762. When the slider 720 reaches the residual position in which the residue sensor 742 is provided, a predetermined alarm is output to an operator (a practitioner or an assistant). The fluid container 760 currently in use is replaced with a new fluid container 760 at an appropriate time determined by the operator. Alternatively, when the second pump 700b having the same configuration as that of the pump 700 (the first pump 700a) is prepared, a switching operation is performed so as to supply the fluid from an auxiliary second pump 700b to the pulsation generator 100.

The first limit sensor 741 indicates a limit position (hereinafter, also referred to as a first limit position) in a movable range in which the slider 720 can move from the home position in the push-in direction of the plunger 762. When the slider 720 reaches the first limit position in which the first limit sensor 741 is provided, the residue of the fluid in the fluid container 760 is much less than the residue indicating that the slider 720 is present at the residual position, and a predetermined alarm is output to the operator. In this case, the fluid container 760 currently in use is also replaced with a new fluid container 760, or a switching operation is also performed so as to supply the fluid from an auxiliary second pump 700b.

In contrast, the second limit sensor 744 indicates a limit position (hereinafter, also referred to as a second limit position) in a movable range in which the slider 720 can move from the home position in the opposite direction of the push-in direction of the plunger 762. When the slider 720 reaches the second limit position in which the second limit sensor 744 is provided, a predetermined alarm is output.

A touch sensor (a contact detection unit) 723 and a pressure sensor (a pressure detection unit) 722 are mounted on the slider 720.

The touch sensor 723 is a sensor for detecting whether the slider 720 is in contact with the plunger 762 of the fluid container 760, that is, the slider 720 is in contact with the fluid accommodation portion 765. The touch sensor 723 outputs an ON signal when the slider 720 is in contact with the plunger 762 of the fluid container 760, and outputs an OFF signal when the slider 720 is not in contact with the plunger 762.

The pressure sensor 722 is a sensor that detects the pressure of the fluid in the fluid accommodation portion 765 formed by the inner wall of the syringe 761 and the gasket 763, that is, a pressure when the slider 720 presses the fluid accommodation portion 765, and outputs signals (detection signals) at a level (for example, a current, or a voltage and a frequency) that corresponds to a detected pressure.

When the pinch valve 750 is closed, and the slider 720 moves in the push-in direction, and after the slider 720 comes into contact with the plunger 762, the pressure of the fluid in the fluid accommodation portion 765 increases to the extent that the slider 720 moves further in the push-in direction.

In contrast, when the pinch valve 750 is opened, and the slider 720 moves in the push-in direction, and even after the slider 720 comes into contact with the plunger 762, the fluid in the fluid accommodation portion 765 flows out of the nozzle 211 of the pulsation generator 100 via the connection tube 25, and thereby the pressure of the fluid in the fluid accommodation portion 765 increases to a certain level, but the pressure of the fluid does not increase even though the slider 720 moves further in the push-in direction.

Signals from the touch sensor 723 and the pressure sensor 722 are input to the pump control unit 710.

In the following description, the slider 720, the motor 730, and the linear guide 740 may be referred to as a fluid pressing unit 731. The fluid pressing unit 731 causes the fluid to flow out of the opening (the fluid outlet) 764 of the fluid container 760 by pressing the fluid accommodation portion 765.

A description to be given hereinafter is regarding a preparation operation configured to include a process of mounting a fluid container 760 filled with the fluid on the fluid container mounting unit 770; a process of supplying the fluid in the fluid container 760 to the pulsation generator 100; and a process of bringing the fluid ejection device 1 into a state in which the pulsation generator 100 can eject the fluid in the form of a pulsed flow.

First, the operator inputs an ON signal of the slider release switch 780 to the pump control unit 710 by operating the slider release switch 780. Thus, the pump control unit 710 moves the slider 720 to the home position.

The operator mounts the fluid container 760 connected to the connection tube 25 in advance on the fluid container mounting unit 770. The syringe 761 of the fluid container 760 is already filled with the fluid.

When the operator sets the connection tube 25 to the pinch valve 750, and then inputs an ON signal of the pinch valve switch 785 to the pump control unit 710 by operating the pinch valve switch 785, the pump control unit 710 closes the pinch valve 750.

Subsequently, the operator inputs an ON signal of the slider set switch 781 to the pump control unit 710 by operating the slider set switch 781. Thus, the pump control unit 710 starts a control operation in such a manner that the slider 720 moves in the push-in direction and the pressure of the fluid accommodated in the fluid accommodation portion 765 of the fluid container 760 becomes a predetermined target pressure value.

Thereafter, when the operator inputs an ON signal of the fluid supply ready switch 782 to the pump control unit 710 by pushing the fluid supply ready switch 782, and the pressure of the fluid in the fluid accommodation portion 765 enters a specific range (hereinafter, also referred to as a rough window) for the target pressure value, the pump control unit 710 is brought into a fluid suppliable state in which the fluid is allowed to be supplied from the pump 700 to the pulsation generator 100.

When the pump control unit 710 is in a fluid suppliable state, and the operator inputs an ON signal of the priming switch 783 to the pump control unit 710 by operating the priming switch 783, the pump control unit 710 starts a priming process. The priming process is a process by which a fluid channel from the fluid container 760 to the connection tube 25 and to a fluid ejection opening 212 of the pulsation generator 100 is filled up with the fluid.

When the priming process starts, the pump control unit 710 opens the pinch valve 750, and starts moving the slider 720 in the push-in direction at the same time or substantially the same time (for example, a time gap of approximately several milliseconds or approximately several tens of milliseconds) as when the pinch valve 750 is opened. The slider 720 moves at a predetermined speed in such a manner that a constant amount of the fluid per unit time is supplied from the fluid container 760. The priming process is performed until a predetermined amount of time required to complete the priming process has elapsed (or the slider 720 moves by a predetermined distance) , or the operator inputs an OFF signal of the priming switch 783 by operating the priming switch 783.

Accordingly, a predetermined amount of the fluid in the fluid accommodation portion 765 is supplied at a predetermined flow speed (the amount of discharge of the fluid per unit time) from the pump 700, the connection tube 25 from the pinch valve 750 to the pulsation generator 100 is filled up with the fluid, and the fluid chamber 501 of the pulsation generator 100, the fluid ejection tube 200 and the like are filled up with the fluid. Air present in the connection tube 25 or the pulsation generator 100 prior to the start of the priming process is released to the atmosphere via the nozzle 211 of the pulsation generator 100 as the fluid flows into the connection tube 25 or the pulsation generator 100.

The pump control unit 710 pre-stores the predetermined speed, the predetermined distance, and the predetermined amount of time in relation to the movement of the slider 720 during the priming process.

As such, the priming process is completed.

Subsequently, when the operator inputs an ON signal of the flushing switch 628 to the drive control unit 600 by operating the flushing switch 628, the drive control unit 600 and the pump control unit 710 start a deaeration process.

The deaeration process is a process by which air bubbles remaining in the connection tube 25 or the pulsation generator 100 are discharged via the nozzle 211 of the pulsation generator 100.

In the deaeration process, in a state in which the pinch valve 750 is opened, the pump control unit 710 moves the slider 720 in the push-in direction at the predetermined speed in such a manner that a constant amount of the fluid per unit time is supplied from the fluid container 760, and the fluid is supplied to the pulsation generator 100. The drive control unit 600 drives the piezoelectric element 401 of the pulsation generator 100 in conjunction with the discharge of the fluid by the pump 700, and thereby the pulsation generator 100 to eject the fluid. Accordingly, air bubbles remaining in the connection tube 25 or the pulsation generator 100 are discharged via the nozzle 211 of the pulsation generator 100. The deaeration process is performed until a predetermined amount of time has elapsed (or the slider 720 moves by a predetermined distance), or the operator inputs an OFF signal of the flushing switch 628 by operating the flushing switch 628.

The drive control unit 600 and the pump control unit 710 pre-store the predetermined speed, the predetermined distance, and the predetermined amount of time in relation to the movement of the slider 720 during the deaeration process.

When the deaeration process is completed, the pump control unit 710 closes the pinch valve 750, and detects the pressure of the fluid accommodated in the fluid accommodation portion 765 of the fluid container 760. The pump control unit 710 performs a control operation in which the position of the slider 720 is adjusted in order for the pressure to become the target pressure value determined corresponding to the intensity of ejection.

Thereafter, when the pressure of the fluid in the fluid accommodation portion 765 enters a specific range (a rough window) for the target pressure value, the pump control unit 710 is brought into a fluid ejectable state in which the fluid can be ejected in the form of a pulsed flow from the pulsation generator 100.

In this state, when the operator inputs an ON signal of the pulsation generator start-up switch 625 to the drive control unit 600 by operating the pulsation generator start-up switch 625 via the feet, the pump control unit 710 opens the pinch valve 750 in response to signals transmitted from the drive control unit 600, and starts the supply of the fluid to the pulsation generator 100 by moving the slider 720 at a predetermined speed in the push-in direction at the same time or substantially the same time (for example, a time gap of approximately several milliseconds or approximately several tens of milliseconds) as when the pinch valve 750 is opened. In contrast, the drive control unit 600 generates a pulsed flow by starting the driving of the piezoelectric element 401 and changing the volume of the fluid chamber 501. Accordingly, a pulsed flow of the fluid is ejected at a high speed via the nozzle 211 at the tip of the pulsation generator 100.

Thereafter, when the operator inputs an OFF signal of the pulsation generator start-up switch 625 to the drive control unit 600 by operating the pulsation generator start-up switch 625 via the feet, the drive control unit 600 stops the driving of the piezoelectric element 401. The pump control unit 710 stops the movement of the slider 720 in response to signals transmitted from the drive control unit 600, and closes the pinch valve 750. As such, the pulsation generator 100 stops the ejection of the fluid.

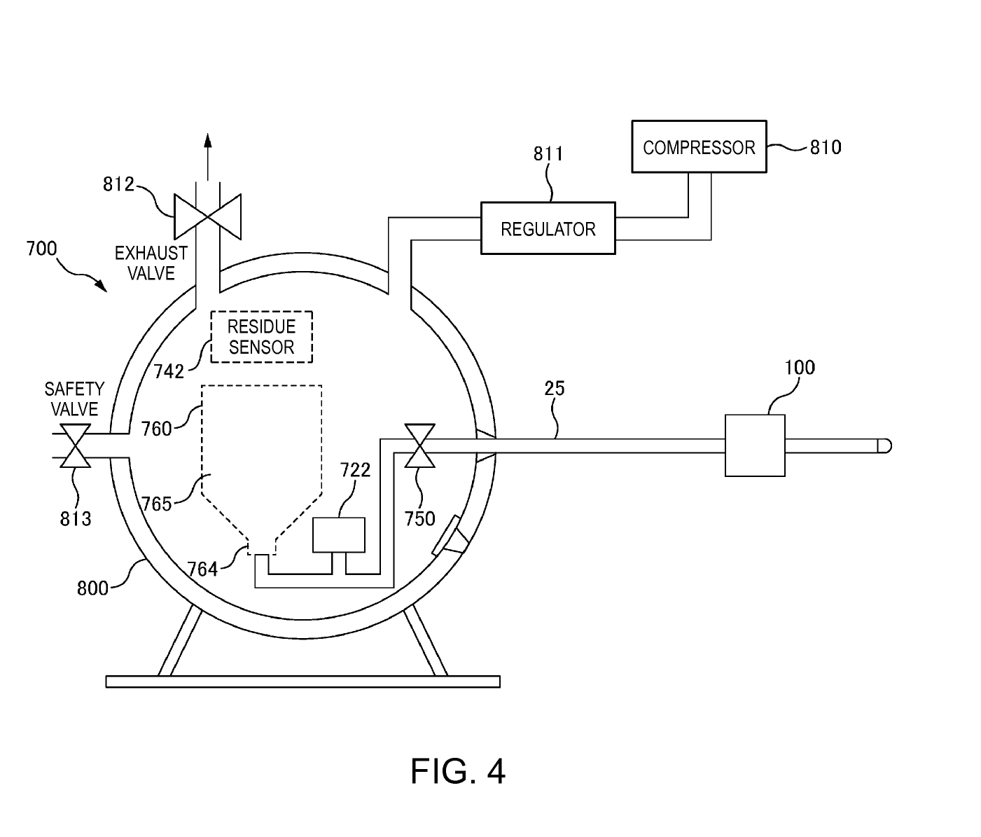

The pump 700 according to the embodiment has a configuration in which the slider 720 presses the fluid container 760 that is formed of a medical syringe includes the syringe 761 and the plunger 762 but may have a configuration as illustrated in FIG. 4.

The pump 700 illustrated in FIG. 4 has a configuration in which the fluid container 760 formed of a transfusion bag is mounted in a pressing chamber 800, and air supplied from a compressor 810 is smoothed by a regulator 811 so as to be pressure-fed to the inside of the pressing chamber 800, thereby pressing the fluid container 760.

When the pinch valve 750 is opened in a state in which the fluid container 760 is pressed by pressing the air in the pressing chamber 800, the fluid accommodated in the fluid accommodation portion 765 of the fluid container 760 flows out of the opening 764 and is supplied to the pulsation generator 100 via the connection tube 25.

The air in the pressing chamber 800 is released to the atmosphere by opening an exhaust valve 812. In a case where the pressure of the air in the pressing chamber 800 exceeds a predetermined pressure, a safety valve 813 is opened and thus the air in the pressing chamber 800 is released to the atmosphere even if the exhaust valve 812 is not opened.

The above-described compressor 810, regulator 811, the exhaust valve 812, the pinch valve 750 are controlled by the pump control unit 710 which is not illustrated in FIG. 4.

A detection signal output from the pressure sensor 722 that detects the pressure of the fluid in the fluid container 760 or the residue sensor 742 that detects a residue of the fluid in the fluid container 760 is also input to the pump control unit 710.

In a case of the pump 700 illustrated in FIG. 4, the compressor 810, the regulator 811, and the pressing chamber 800 form the fluid pressing unit 731.

By employing the pump 700 according to such an aspect, it is possible to increase an amount of the fluid which can be supplied to the pulsation generator 100 per unit time. Since the fluid can be supplied to the pulsation generator 100 at a high pressure, a transfusion bag that accommodates the fluid is used as the fluid container 760 as it is, and thus it is possible to prevent the fluid from being contaminated. It is possible to continuously supply the fluid without the occurrence of pulsation in the pulsation generator 100.

In addition, in the embodiment, the drive control unit 600 is provided separately from the pump 700 and the pulsation generator 100; however, the drive control unit 600 may be provided integrally with the pump 700.

When the practitioner performs an operation using the fluid ejection device 1, the practitioner grasps the pulsation generator 100. Accordingly, the connection tube 25 up to the pulsation generator 100 is preferably as flexible as possible. For this reason, a flexible thin tube is used as the connection tube 25, and a fluid discharge pressure of the pump 700 is preferably set to a low pressure in a pressure range in which the fluid can be supplied to the pulsation generator 100. For this reason, the discharge pressure of the pump 700 is set to approximately 0.3 atm (0.03 MPa) or less.

In particular, in a case where a malfunction of an apparatus may lead to a serious accident, for example, for a brain surgery, it is necessary to prevent the cutting of the connection tube 25 from causing the ejection of the fluid at a high pressure, and also, for this reason, the discharge pressure of the pump 700 is required to be set to a low pressure.

### Pulsation Generator

Subsequently, the structure of the pulsation generator 100 according to the embodiment will be described.

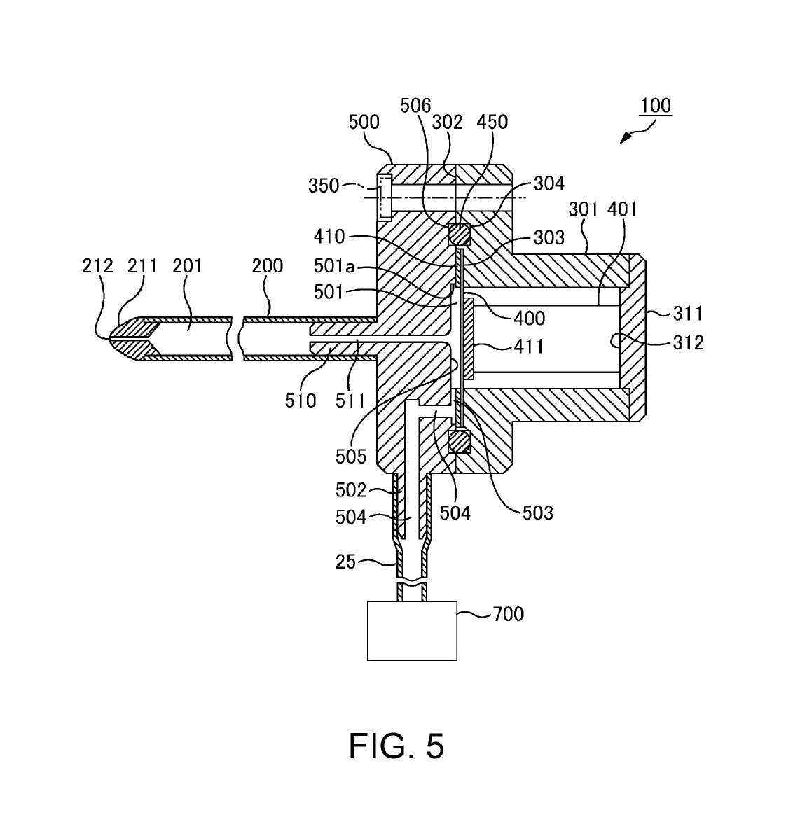

FIG. 5 is a cross-sectional view illustrating the structure of the pulsation generator 100 according to the embodiment. In FIG. 5, the pulsation generator 100 includes a pulse generation unit that generates the pulsation of the fluid, and is connected to the fluid ejection tube 200 having a connection channel 201 as a channel through which the fluid is discharged.

In the pulsation generator 100, an upper case 500 and a lower case 301 are screwed together with four fixation screws 350 (not illustrated) while the respective facing surfaces thereof are bonded to each other. The lower case 301 is a cylindrical member having a flange, and one end portion of the lower case 301 is sealed with a bottom plate 311. The piezoelectric element 401 is provided in an inner space of the lower case 301.

The piezoelectric element 401 is a stack-type piezoelectric element, and acts as an actuator. One end portion of the piezoelectric element 401 is firmly fixed to the diaphragm 400 via an upper plate 411, and the other end portion is firmly fixed to an upper surface 312 of the bottom plate 311.

The diaphragm 400 is made of a circular disc-like thin metal plate, and a circumferential edge portion of the diaphragm 400 is firmly fixed to a bottom surface of a concave portion 303 in the lower case 301 while being in close contact with the bottom surface of the concave portion 303. When drive signals are input to the piezoelectric element 401 that acts as a volume change unit, the piezoelectric element 401 changes the volume of the fluid chamber 501 via the diaphragm 400 through the extension and contraction thereof.

A reinforcement plate 410 is provided in such a manner as to be stacked on an upper surface of the diaphragm 400, and is made of a circular disc-like thin metal plate having an opening at the center thereof.

The upper case 500 has a concave portion formed in a center portion of the surface facing the lower case 301, and the fluid chamber 501 is a rotator-shaped space formed by this concave portion and the diaphragm 400 and filled with the fluid. That is, the fluid chamber 501 is a space enveloped by a sealing surface 505 and an inner side wall 501a of the concave portion of the upper case 500, and the diaphragm 400. An outlet channel 511 is drilled in an approximately center portion of the fluid chamber 501.

The outlet channel 511 passes through the outlet channel tube 510 from the fluid chamber 501 to an end portion of an outlet channel tube 510 provided in such a manner as to protrude from one end surface of the upper case 500. A connection portion between the outlet channel 511 and the sealing surface 505 of the fluid chamber 501 is smoothly rounded so as to reduce fluid resistance.

In the embodiment (refer to FIG. 5) , the fluid chamber 501 has a substantially cylindrical shape having sealed opposite ends; however, the fluid chamber 501 may have a conical shape, a trapezoidal shape, a hemispherical shape, or the like in a side view, and the shape of the fluid chamber 501 is not limited to a cylindrical shape. For example, when the connection portion between the outlet channel 511 and the sealing surface 505 has a funnel shape, air bubbles in the fluid chamber 501 (to be described later) are easily discharged.

The fluid ejection tube 200 is connected to the outlet channel tube 510. The connection channel 201 is drilled in the fluid ejection tube 200, and the diameter of the connection channel 201 is larger than that of the outlet channel 511. In addition, the tube thickness of the fluid ejection tube 200 is formed so as to have a range of rigidity in which the fluid ejection tube 200 does not absorb pressure pulsation of the fluid.

The nozzle 211 is inserted into the tip end portion of the fluid ejection tube 200. A fluid ejection opening 212 is drilled in the nozzle 211. The diameter of the fluid ejection opening 212 is smaller than that of the connection channel 201.

An inlet channel tube (a fluid intake port) 502 is provided in such a manner as to protrude from a side surface of the upper case 500, and is inserted into the connection tube 25 through which the fluid is supplied from the pump 700. A connection channel 504 for the inlet channel is drilled in the inlet channel tube 502. The connection channel 504 communicates with an inlet channel 503. The inlet channel 503 is formed in a groove shape in a circumferential edge portion of the sealing surface 505 of the fluid chamber 501, and communicates with the fluid chamber 501.

A packing box 304 and a packing box 506 are respectively formed in the bonded surfaces of the lower case 301 and the upper case 500 at positions separated from an outer circumferential direction of the diaphragm 400, and a ring-shaped packing 450 is mounted in a space formed by the packing boxes 304 and 506.

Here, when the upper case 500 and the lower case 301 are assembled together, the circumferential edge portion of the diaphragm 400 is in close contact with a circumferential edge portion of the reinforcement plate 410 due to the circumferential edge portion of the sealing surface 505 of the upper case 500 and the bottom surface of the concave portion 303 of the lower case 301. At this time, the packing 450 is pressed by the upper case 500 and the lowercase 301, and thereby the fluid is prevented from leaking from the fluid chamber 501.

Since the inner pressure of the fluid chamber 501 becomes a high pressure of 30 atm (3 MPa) or greater during the discharge of the fluid, the fluid may slightly leak from the respective connections between the diaphragm 400, the reinforcement plate 410, the upper case 500, and the lower case 301; however, the leakage of the fluid is prevented due to the packing 450.

As illustrated in FIG. 5, in the case where the packing 450 is provided, since the packing 450 is compressed due to the pressure of the fluid leaking from the fluid chamber 501 at a high pressure, and is strongly pressed against the respective walls of the packing boxes 304 and 506, it is possible to more reliably prevent the leakage of the fluid. For this reason, it is possible to maintain a considerable increase in the inner pressure of the fluid chamber 501 during the driving of the pulsation generator 100.

Subsequently, the inlet channel 503 formed in the upper case 500 will be described with reference to the drawings in more detail.

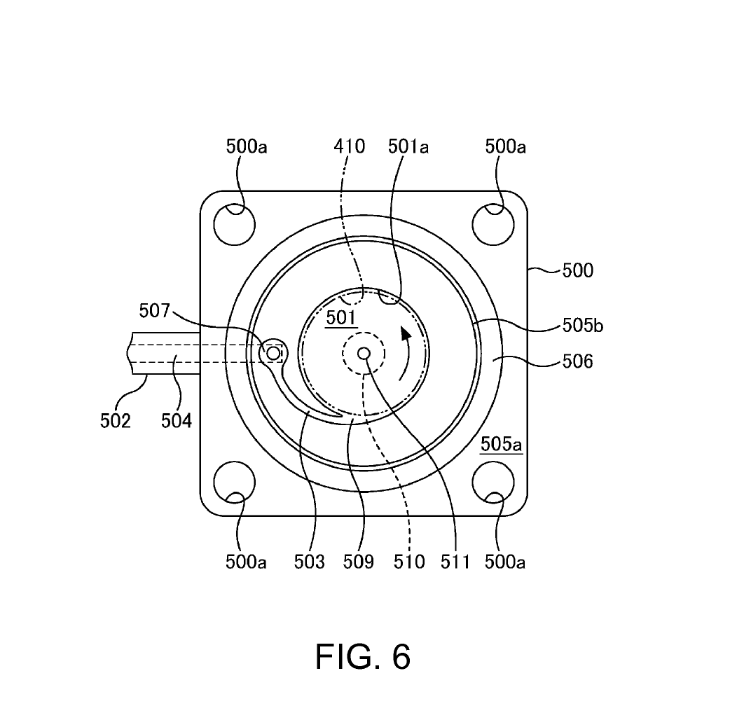

FIG. 6 is a plan view illustrating the shape of the inlet channel 503, and illustrates the shape of the upper case 500 when the surface of the upper case 500 bonded to the lower case 301 is seen.

In FIG. 6, the inlet channel 503 is formed in a groove shape in the circumferential edge portion of the sealing surface 505 of the upper case 500.

One end portion of the inlet channel 503 communicates with the fluid chamber 501, and the other end portion communicates with the connection channel 504. A fluid sump 507 is formed in a connection portion between the inlet channel 503 and the connection channel 504. A connection portion between the fluid sump 507 and the inlet channel 503 is smoothly rounded, and thereby fluid resistance is reduced.

The inlet channel 503 communicates with the fluid chamber 501 in a substantially tangential direction with respect to an inner circumferential side wall 501a of the fluid chamber 501. The fluid supplied from the pump 700 (refer to FIG. 1) at a predetermined pressure flows along the inner circumferential side wall 501a (in a direction illustrated by the arrow in FIG. 6), and generates a swirl flow in the fluid chamber 501. The swirl flow is pushed against the inner circumferential side wall 501a due to a centrifugal force associated with the swirling of the fluid, and air bubbles in the fluid chamber 501 are concentrated in a center portion of the swirl flow.

The air bubbles concentrated in the center portion are discharged via the outlet channel 511. For this reason, the outlet channel 511 is preferably provided in the vicinity of the center of the swirl flow, that is, in an axial center portion of a rotor shape.

As illustrated in FIG. 6, the inlet channel 503 is curved. The inlet channel 503 may communicate with the fluid chamber 501 while not being curved but being linearly formed; however, when the inlet channel 503 is curved, a channel length is increased, and a desired inertance (to be described later) is obtained in a small space.

As illustrated in FIG. 6, the reinforcement plate 410 is provided between the diaphragm 400 and the circumferential edge portion of the sealing surface 505, in which the inlet channel 503 is formed. The reinforcement plate 410 is provided so as to improve the durability of the diaphragm 400. Since a cut-out connection opening 509 is formed in a connection portion between the inlet channel 503 and the fluid chamber 501, when the diaphragm 400 is driven at a high frequency, stress may be concentrated in the vicinity of the connection opening 509, and thereby a fatigue failure may occur in the vicinity of the connection opening 509. It is possible to prevent stress from being concentrated on the diaphragm 400 by providing the reinforcement plate 410 with an opening not having a cut-out portion and being continuously formed.

Four screw holes 500a are respectively provided in outer circumferential corner portions of the upper case 500, and the upper case 500 and the lower case 301 are bonded to each other via screwing at the positions of the screw holes.

It is possible to firmly fix the reinforcement plate 410 and the diaphragm 400 in an integrally stacked state by bonding together the reinforcement plate 410 and the diaphragm 400, which is not illustrated. An adhesion method using an adhesive, a solid-state diffusion bonding method, a welding method, or the like may be used so as to firmly fix together the reinforcement plate 410 and the diaphragm 400; however, the respective bonded surfaces of the reinforcement plate 410 and the diaphragm 400 are preferably in close contact with each other.

### Operation of Pulsation Generator

Subsequently, an operation of the pulsation generator 100 according to the embodiment will be described with reference to FIGS. 1 to 6. The pulsation generator 100 according to the embodiment discharges the fluid due to a difference between an inertance L1 (may be referred to as a combined inertance L1) of the inlet channel 503 and the peripherals and an inertance L2 (may be referred to as a combined inertance L2) of the outlet channel 511 and the peripherals.

### Inertance

First, the inertance will be described.

An inertance L is expressed by L=ρ×h/S, and here, ρis the density of a fluid, S is the cross-sectional area of a channel, and h is a channel length. When ΔP is a differential pressure of the channel, and Q is a flow rate of the fluid flowing through the channel, it is possible to deduce a relationship ΔP=L×dQ/dt by modifying an equation of motion in the channel using the inertance L.

That is, the inertance L indicates a degree of influence on a change in flow rate with time, and a change in flow rate with time decreases to the extent that the inertance L is large, and a change in flow rate with time increases to the extent that the inertance L is small.

Similar to a parallel connection or a series connection of inductances in an electric circuit, it is possible to calculate a combined inertance with respect to a parallel connection of a plurality of channels or a series connection of a plurality of channels having different shapes by combining an inertance of each of the channels.

Since the diameter of the connection channel 504 is set to be larger much than that of the inlet channel 503, the inertance L1 of the inlet channel 503 and the peripherals can be calculated from a boundary of the inlet channel 503. At this time, since the connection tube 25 that connects the pump 700 and the inlet channel 503 is flexible, the connection tube 25 may not be taken into consideration in calculating the inertance L1.

Since the diameter of the connection channel 201 is larger much than that of the outlet channel 511, and the tube (tube wall) thickness of the fluid ejection tube 200 is thin, the connection tube 25 and the fluid ejection device 1 have a negligible influence on the inertance L2 of the outlet channel 511 and the peripherals. Accordingly, the inertance L2 of the outlet channel 511 and the peripherals may be replaced with an inertance of the outlet channel 511.

The rigidity of the tube wall thickness of the fluid ejection tube 200 is sufficient to propagate the pressure of the fluid.

In the embodiment, a channel length and a cross-sectional area of the inlet channel 503 and a channel length and a cross-sectional area of the outlet channel 511 are set in such a manner that the inertance L1 of the inlet channel 503 and the peripherals is greater than the inertance L2 of the outlet channel 511 and the peripherals.

### Ejection of Fluid

Subsequently, an operation of the pulsation generator 100 will be described.

The pump 700 supplies the fluid to the inlet channel 503 at a predetermined pressure. As a result, when the piezoelectric element 401 is not operated, the fluid flows into the fluid chamber 501 due to a difference between a discharge force of the pump 700 and a fluid resistance value for the entirety of the inlet channel 503 and the peripherals.

Here, in a case where the inertance L1 of the inlet channel 503 and the peripherals and the inertance L2 of the outlet channel 511 and the peripherals are considerably large, when a drive signal is input to the piezoelectric element 401, and the piezoelectric element 401 extends rapidly, the inner pressure of the fluid chamber 501 increases rapidly, and reaches several tens of atmosphere.

Since the inner pressure of the fluid chamber 501 is larger much than the pressure applied to the inlet channel 503 by the pump 700, the flow of the fluid from the inlet channel 503 to the fluid chamber 501 decreases due to the pressure, and the flow of the fluid out of the outlet channel 511 increases.

Since the inertance L1 of the inlet channel 503 is larger than the inertance L2 of the outlet channel 511, an increase in a flow rate of the fluid discharged from the outlet channel 511 is larger than a decrease in a flow rate of the fluid flowing from the inlet channel 503 into the fluid chamber 501. Accordingly, the fluid is discharged in the form of a pulsed flow to the connection channel 201, that is, a pulsed flow occurs. Discharge pressure fluctuation propagates in the fluid ejection tube 200, and the fluid is ejected via the fluid ejection opening 212 of the nozzle 211 at the tip end.

Here, since the diameter of the fluid ejection opening 212 of the nozzle 211 is smaller than that of the outlet channel 511, a pulsed flow of the fluid is ejected as droplets at a higher pressure and speed.

In contrast, immediately after a pressure increase, the inner pressure of the fluid chamber 501 becomes negative due to interaction between a decrease in the amount of inflow of the fluid from the inlet channel 503 and an increase in the amount of outflow of the fluid from the outlet channel 511. As a result, after a predetermined amount of time has elapsed, due to both of the pressure of the pump 700 and the negative inner pressure of the fluid chamber 501, the fluid flows from the inlet channel 503 into the fluid chamber 501 again at the same speed as that before the operation of the piezoelectric element 401.

When the piezoelectric element 401 extends after the outflow of the fluid from the inlet channel 503 is restored, it is possible to continuously eject the fluid in the form of a pulsed flow via the nozzle 211.

### Discharge of Air Bubbles

Subsequently, an operation of discharging air bubbles from the fluid chamber 501 will be described.

As described above, the inlet channel 503 communicates with the fluid chamber 501 via a path that approaches the fluid chamber 501 while swirling around the fluid chamber 501. The outlet channel 511 is provided in the vicinity of a rotational axis of a substantially rotor-shaped fluid chamber 501.

For this reason, the fluid flowing from the inlet channel 503 into the fluid chamber 501 swirls along the inner circumferential side wall 501a of the fluid chamber 501. The fluid is pushed against the inner circumferential side wall 501a of the fluid chamber 501 due to a centrifugal force, and air bubbles contained in the fluid are concentrated in the center portion of the fluid chamber 501, and are discharged via the outlet channel 511.

Accordingly, even when a small amount of the volume of the fluid chamber 501 is changed in association with the operation of the piezoelectric element 401, it is possible to obtain a sufficient pressure increase while a pressure fluctuation is not adversely affected by air bubbles.

In the embodiment, since the pump 700 supplies the fluid to the inlet channel 503 at a predetermined pressure, even when the driving of the pulsation generator 100 is stopped, the fluid is supplied to the inlet channel 503 and the fluid chamber 501. Accordingly, it is possible to start an initial operation without an aid of a prime operation.

Since the fluid is ejected via the fluid ejection opening 212 having a diameter smaller than that of the outlet channel 511, an inner fluid pressure is increased higher than that of the outlet channel 511, and thereby it is possible to eject the fluid at a high speed.

Since the rigidity of the fluid ejection tube 200 is sufficient to transmit a pulsation of the fluid from the fluid chamber 501 to the fluid ejection opening 212, it is possible to eject the fluid in the form of a desired pulsed flow without disturbing pressure propagation of the fluid from the pulsation generator 100.

Since the inertance of the inlet channel 503 is set to be larger than that of the outlet channel 511, an increase in the amount of outflow of the fluid from the outlet channel 511 is larger than a decrease in the amount of flow of the fluid from the inlet channel 503 into the fluid chamber 501, and it is possible to discharge the fluid into the fluid ejection tube 200 in the form of a pulsed flow. Accordingly, a check valve is not required to be provided in the inlet channel 503, it is possible to simplify the structure of the pulsation generator 100, it is easy to clean the inside of the pulsation generator 100, and it is possible to remove a potential durability problem associated with the use of the check valve.

Since the respective inertances of both of the inlet channel 503 and the outlet channel 511 are set to be considerably large, it is possible to rapidly increase the inner pressure of the fluid chamber 501 by rapidly reducing the volume of the fluid chamber 501.

Since the piezoelectric element 401 as a volume change unit and the diaphragm 400 are configured so as to generate a pulsation, it is possible to simplify the structure of the pulsation generator 100 and to reduce the size of the pulsation generator 100 in association therewith. It is possible to set the maximum frequency of a change in the volume of the fluid chamber 501 to a high frequency of 1 KHz or greater, and the pulsation generator 100 is optimized to eject a pulsed flow of the fluid at a high speed.

In the pulsation generator 100, since the inlet channel 503 generates a swirl flow of the fluid in the fluid chamber 501, the fluid in the fluid chamber 501 is pushed in an outer circumferential direction of the fluid chamber 501 due to a centrifugal force, air bubbles contained in the fluid are concentrated in the center portion of the swirl flow, that is, in the vicinity of the axis of the substantially rotor shape, and thereby it is possible to discharge the air bubbles via the outlet channel 511 provided in the vicinity of the axis of the substantially rotor shape. For this reason, it is possible to prevent a decrease in pressure amplitude associated with the stagnation of air bubbles in the fluid chamber 501, and it is possible to continuously and stably drive the pulsation generator 100.

Since the inlet channel 503 is formed in such a manner as to communicate with the fluid chamber 501 via the path that approaches the fluid chamber 501 while swirling around the fluid chamber 501, it is possible to generate a swirl flow without adopting a structure dedicated for swirling the fluid in the fluid chamber 501.

Since the groove-shaped inlet channel 503 is formed in the outer circumferential edge portion of the sealing surface 505 of the fluid chamber 501, it is possible to form the inlet channel 503 (a swirl flow generation unit) without increasing the number of components.

Since the reinforcement plate 410 is provided on the upper surface of the diaphragm 400, the diaphragm 400 is driven with respect to an outer circumference (a fulcrum) of the opening of the reinforcement plate 410, and thereby the concentration of stress is unlikely to occur, and it is possible to improve the durability of the diaphragm 400.

When corners of the bonded surface of the reinforcement plate 410 bonded to the diaphragm 400 is rounded, it is possible to further reduce the concentration of stress on the diaphragm 400.

When the reinforcement plate 410 and the diaphragm 400 are firmly and integrally fixed together while being stacked on each other, it is possible to improve the assemblability of the pulsation generator 100, and it is possible to reinforce the outer circumferential edge portion of the diaphragm 400.

Since the fluid sump 507 for the stagnation of the fluid is provided in the connection portion between the connection channel 504 on an inlet side for supplying the fluid from the pump 700 and the inlet channel 503, it is possible to prevent the inertance of the connection channel 504 from affecting the inlet channel 503.

In the respective bonded surfaces of the lower case 301 and the upper case 500, the ring-shaped packing 450 is provided at the position separated from the outer circumferential direction of the diaphragm 400, and thereby it is possible to prevent the leakage of the fluid from the fluid chamber 501, and to prevent a decrease in the inner pressure of the fluid chamber 501.

### Detection of Failure in Pressure Sensor

As described above, in the fluid ejection device 1 according to the embodiment, in a case where the inner fluid pressure of the fluid accommodation portion 765 enters a specific range (rough window) for the predetermined target pressure value, and a fluid suppliable state occurs in which the fluid is allowed to be supplied from the pump 700 to the pulsation generator 100, when the practitioner inputs an ON signal of the pulsation generator start-up switch 625 to the drive control unit 600, the drive control unit 600 starts driving of the piezoelectric element 401, and thus a pulsed flow of the fluid is ejected at a high speed via the nozzle 211 at the tip of the pulsation generator 100.

As mentioned above, the fluid ejection device 1 performs control so that the inner fluid pressure of the fluid accommodation portion 765 enters the rough window, and thus ejection of the fluid from the pulsation generator 100 is performed at an appropriate intensity.

Therefore, in a case where the pressure sensor 722 that detects the inner fluid pressure of the fluid accommodation portion 765 fails for unknown reasons, the inner fluid pressure of the fluid accommodation portion 765 cannot be controlled, and thus there is a possibility that the fluid may be ejected from the pulsation generator 100 at an intensity which cannot be expected by the practitioner.

In the embodiment, a failure in the pressure sensor 722 indicates a case where output characteristics of the pressure sensor 722 are irregular.

The regular output characteristics of the pressure sensor 722 indicates characteristics in which a correspondence relationship between a pressure applied to the pressure sensor 722 and a level of a detection signal output from the pressure sensor 722 is within a predetermined range which is regarded to be normal.

The fluid ejection device 1 according to the embodiment can detect a failure in the pressure sensor 722.

As will be described later in detail, the fluid ejection device 1 according to the embodiment determines that the pressure sensor 722 has failed, for example, in a case where the slider (a moving body) 720 moves in the pressing direction to press the fluid accommodation portion 765 in a state in which the pinch valve (a channel opening and closing unit) 750 is closed, if a pressure detected by the pressure sensor 722 when the slider 720 comes into contact with the plunger 762 is lower than a first determination value, or if a pressure detected by the pressure sensor 722 when the slider 720 does not come into contact with the plunger 762 is equal to or higher than a second determination value.

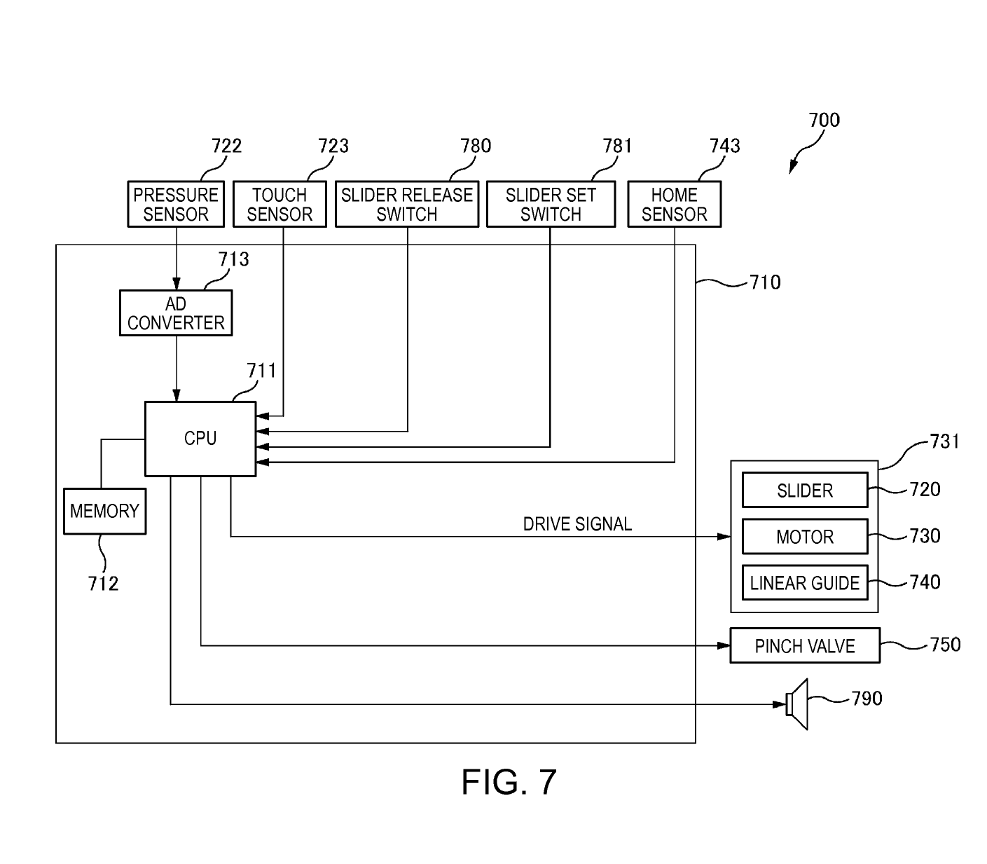

Hereinafter, with reference to FIGS. 7 to 12, a detailed description will be made of a failure detection process of the pressure sensor 722 performed by the fluid ejection device 1 according to the embodiment.

First, the configuration of the pump control unit (the control unit) 710 will be described with reference to FIG. 7.

The pump control unit 710 is configured to have a central processing unit (CPU) 711, a memory 712, and an analog/digital (AD) converter 713.

The pump control unit 710 takes from the pressure sensor 722 a detection signal corresponding to the inner pressure when the fluid pressing unit 731 presses the fluid accommodation portion 765 of the fluid container 760, and the pump control unit 710 controls the fluid pressing unit 731. For example, when the pump control unit 710 receives an ON signal of the slider set switch 781, the pump control unit 710 controls a pressure in the fluid accommodation portion 765 detected by the pressure sensor 722 to approach the target pressure value by outputting a predetermined drive signal to the fluid pressing unit 731 and driving the motor 730 so as to move the slider 720 in the push-in direction, in a state in which the pinch valve 750 is closed. The fluid pressing unit 731 is configured to have the slider 720, the motor 730, and the linear guide 740.

The CPU 711 controls the entirety of the pump control unit 710, and realizes various functions of the embodiment by executing a program stored in the memory 712 and coded to perform various operations.

The memory 712 stores various pieces of data other than the program. For example, the memory 712 stores target pressure value, and also stores first determination value level data (for example, a voltage value) indicating a level (for example, a voltage) corresponding to the first determination value (a pressure value) , second determination value level data indicating a level corresponding to the second determination value, and the like.

The AD converter 713 receives a detection signal output from the pressure sensor 722, and outputs data indicative of a level of the detection signal. Specifically, the pressure sensor 722 detects a pressure when the slider 720 presses the fluid accommodation portion 765, and outputs a detection signal with a level (for example, a voltage) corresponding to the pressure. The AD converter 713 outputs detected level data (for example, a voltage value) indicative of the level of the detection signal output from the pressure sensor 722.

### First Failure Detection Process

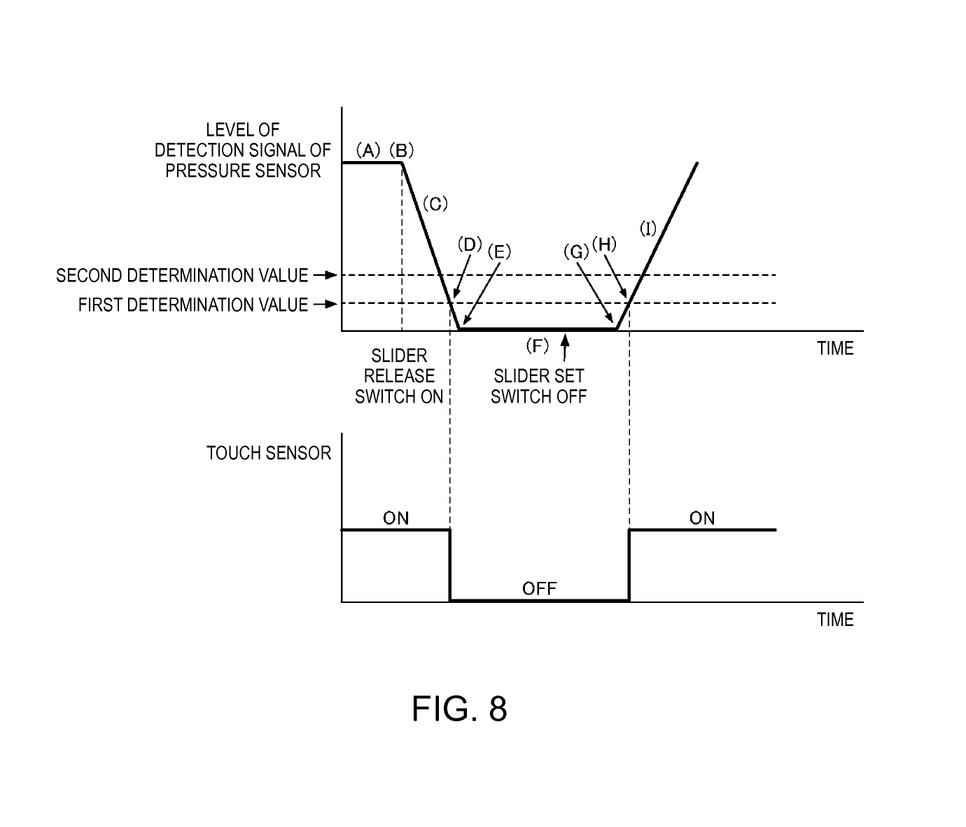

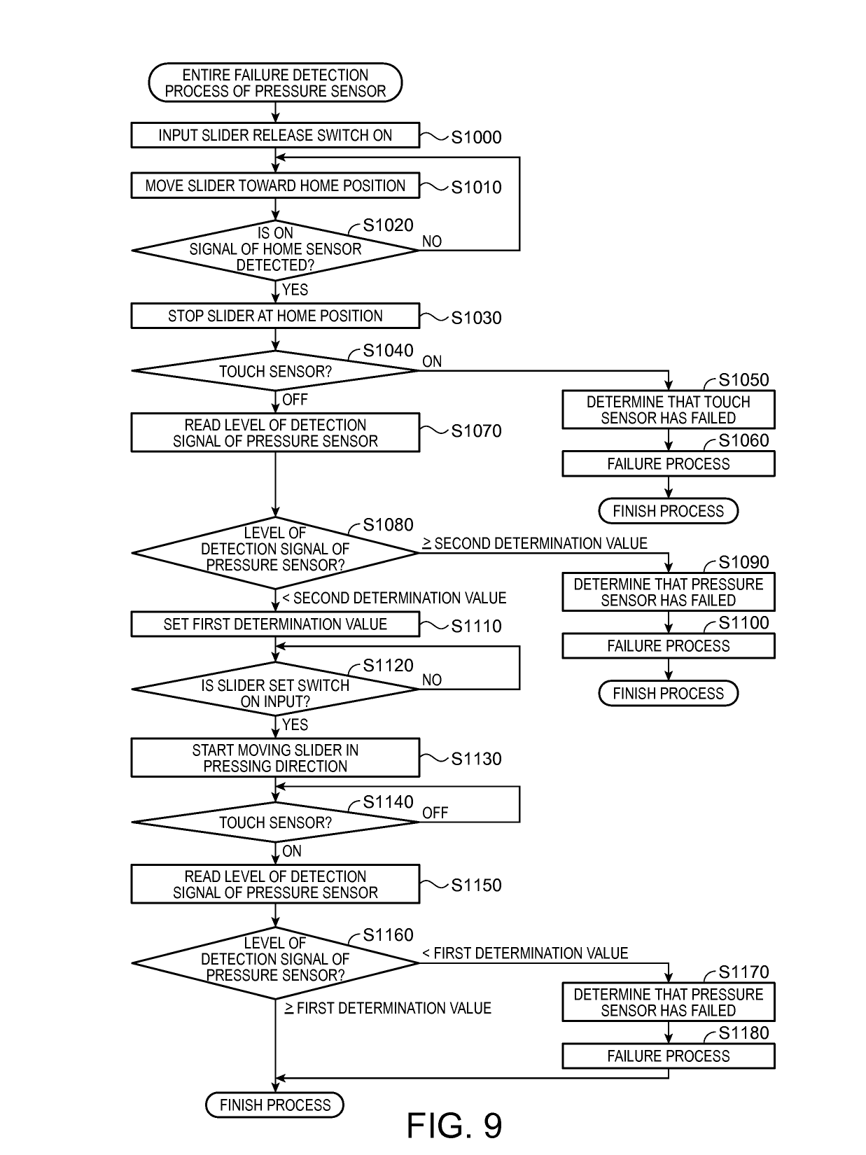

Hereinafter, with reference to FIGS. 8 and 9, a description will be made of a flow of a first failure detection process of the pressure sensor 722 according to the embodiment. FIG. 8 is a diagram illustrating a state in which a level of a detection signal of the pressure sensor 722 and an output signal of the touch sensor 723 are changed, and FIG. 9 is a flowchart illustrating a flow of the first failure detection process.

In the following description, the flow of the first failure detection process will be made from the time at which an ON signal of the slider release switch 780 is input to the pump control unit 710.

First, the CPU 711 detects that an ON signal of the slider release switch 780 has been input ((B) in FIG. 8; S1000 in FIG. 9). Thus, the CPU 711 outputs a predetermined drive signal to the fluid pressing unit 731 so as to move the slider 720 to the home position in the opposite direction to the push-in direction (S1010 in FIG. 9). For this reason, the plunger 762 is moved by the inner fluid pressure of the fluid accommodation portion 765 in the opposite direction to the push-in direction, and thus the inner fluid pressure of the fluid accommodation portion 765 decreases ((C) in FIG. 8).

If the slider 720 separates from the plunger 762 while the CPU 711 moves the slider 720 to the home position, an output signal from the touch sensor 723 is changed to an OFF state ((D) in FIG. 8), and thus the decrease in the inner fluid pressure of the fluid accommodation portion 765 stops ((E) in FIG. 8).

If the CPU 711 subsequently and continuously moves the slider 720 to the home position in the opposite direction to the push-in direction, the slider 720 reaches the position of the home sensor 743. At this time, the CPU 711 detects an ON signal output from the home sensor 743. The CPU 711 stops the slider 720 at the home position ((F) in FIG. 8; S1030 in FIG. 9).

At this point, the CPU 711 determines whether a signal output from the touch sensor 723 is an ON signal or an OFF signal (S1040 in FIG. 9). Since the slider 720 stops at the home position at this point, the CPU 711 determines that the touch sensor 723 has failed (S1050 in FIG. 9) if the signal output from the touch sensor 723 is an ON signal, and performs a failure process (S1060 in FIG. 9).

The CPU 711 outputs, for example, a predetermined alarm in the failure process. For example, the CPU 711 outputs a voice message indicating that the touch sensor 723 has failed from the speaker 790. Alternatively, the CPU 711 turns on a predetermined alarm lamp (not illustrated).

In the above-described manner, it is possible to promptly notify an operator such as a practitioner that the touch sensor 723 has failed and thus to further improve the safety of the fluid ejection device 1.

If the signal output from the touch sensor 723 is an OFF signal in step S1040 of FIG. 9, the CPU 711 acquires detected level data (a voltage value) indicating a level (a voltage) of a detection signal output from the pressure sensor 722, from the AD converter 713 (S1070 in FIG. 9).

The CPU 711 determines whether or not the detected level data is equal to or greater than the second determination value level data (S1080 in FIG. 9).

Since, at this point, the slider 720 is located at the home position and thus does not come into contact with the plunger 762, and the inner pressure of the fluid accommodation portion 765 is a low pressure (a pressure lower than a pressure indicated by the second determination value which will be described later), the pressure sensor 722 does not accurately detect the inner pressure of the fluid accommodation portion 765 if the detected level data is equal to or greater than the second determination value level data.

For this reason, if the detected level data is equal to or greater than the second determination value level data, the CPU 711 determines that the pressure sensor 722 has failed (S1090 in FIG. 9) and performs a predetermined failure process (S1100 in FIG. 9).

The second determination value may be set to an appropriate value through pre-tests or the like. For example, the inner pressure of the fluid accommodation portion 765 obtained when an output signal from the touch sensor 723 switches between an ON state and an OFF state may be measured, and the second determination value may be set on the basis of the pressure at this time. For example, a value obtained by adding a predetermined value (a second predetermined value) to the pressure obtained when an output signal from the touch sensor 723 switches between an ON state and an OFF state, may be set as the second determination value.

If the second determination value is set in the above-described manner, in a case where the inner pressure of the fluid accommodation portion 765 is decreasing, the inner pressure of the fluid accommodation portion 765 can be made lower than the second determination value before the output signal from the touch sensor 723 switches from an ON state to an OFF state, and in a case where the inner pressure of the fluid accommodation portion 765 is increasing, the inner pressure of the fluid accommodation portion 765 can be made higher than the second determination value after the output signal from the touch sensor 723 switches from an OFF state to an ON state. Thus, it is possible to prevent detection errors of a failure in the pressure sensor 722.

If it is determined that the pressure sensor 722 has failed, the CPU 711 outputs a predetermined alarm in the failure process (S1100 in FIG. 9). For example, the CPU 711 outputs a voice message indicating that the pressure sensor 722 has failed from the speaker 790. Alternatively, the CPU 711 turns on a predetermined alarm lamp (not illustrated).

In the above-described manner, it is possible to promptly notify an operator such as a practitioner that the pressure sensor 722 has failed and thus to further improve the safety of the fluid ejection device 1.

If the detected level data of the pressure sensor 722 is smaller than the second determination value level data in step S1080 of FIG. 9, the CPU 711 sets first determination value level data (a voltage value) indicating the first determination value (a pressure value) (S1110 in FIG. 9).

The first determination value is a determination value used to determine a failure in the pressure sensor 722 when the slider 720 comes into contact with the plunger 762 in the following subsequent processes.

In the same manner as in the second determination value, the inner pressure of the fluid accommodation portion 765 obtained when an output signal from the touch sensor 723 switches between an ON state and an OFF state may be measured, and the first determination value may be set on the basis of the pressure at this time. For example, a value obtained by subtracting a first predetermined value from the pressure obtained when an output signal from the touch sensor 723 switches between an ON state and an OFF state, may be set as the first determination value.

If the first determination value is set in the above-described manner, in a case where the inner pressure of the fluid accommodation portion 765 is decreasing, the inner pressure of the fluid accommodation portion 765 can be made lower than the first determination value after the output signal from the touch sensor 723 switches from an ON state to an OFF state, and in a case where the inner pressure of the fluid accommodation portion 765 is increasing, the inner pressure of the fluid accommodation portion 765 can be made higher than the first determination value before the output signal from the touch sensor 723 switches from an OFF state to an ON state. Thus, it is possible to prevent detection errors of a failure in the pressure sensor 722.

Alternatively, since a level of the detection signal of the pressure sensor 722 may vary depending on temperatures or manufacturing errors, the first determination value may be set to a relative value with respect to a level of the detection signal output from the pressure sensor 722 when the slider 720 stops at the home position. In this case, even if there are variations in the output characteristics of the pressure sensor 722, it is possible to prevent detection errors of a failure in the pressure sensor 722.

In the embodiment, the first determination value is a value obtained by adding a third predetermined value to a pressure indicated by the detection signal output from the pressure sensor 722 when the slider 720 is located at the home position (that is, when the slider 720 is located at a predetermined position so as not to come into contact with the fluid accommodation portion 765). In the embodiment, the third predetermined value is a value obtained by additionally adding a positive value which is as small as possible to the maximum error which may be included in the detection signal output from the pressure sensor 722.

In the above-described manner, it is possible to accurately detect the abnormality of a level of the detection signal of the pressure sensor 722 when the touch sensor 723 is in an ON state while removing the influences of errors of the pressure sensor 722.

The third determination value used to calculate the first determination value is more preferably set in consideration of not only errors of the pressure sensor 722 but also errors (for example, a quantization error) of the AD converter 713.

The CPU 711 stores the first determination value level data indicating the first determination value which is set as mentioned above, in the memory 712 (S1110 in FIG. 9).

Then, the CPU 711 detects that an ON signal of the slider set switch 781 has been input (S1120 in FIG. 9). Thus, the CPU 711 outputs a predetermined drive signal to the fluid pressing unit 731 so as to move the slider 720 in the push-in direction (S1130 in FIG. 9).

The CPU 711 determines whether a signal output from the touch sensor 723 is an ON signal or an OFF signal (S1140 in FIG. 9). When the slider 720 comes into contact with the plunger 762 and thus starts pressing the plunger 762 ((G) in FIG. 8), an ON signal is output from the touch sensor 723 ((H) in FIG. 8).

Therefore, the CPU 711 detects that the ON signal has been output from the touch sensor 723, and acquires detected level data (a voltage value) indicating a level (voltage) of the detection signal output from the pressure sensor 722, from the AD converter 713 (S1150 in FIG. 9).

The CPU 711 determines whether or not the detected level data is equal to or greater than the first determination value level data (S1160 in FIG. 9).

Since, at this point, the slider 720 starts pressing the plunger 762, and thus the inner pressure of the fluid accommodation portion 765 is higher than the pressure indicated by the first determination value, the pressure sensor 722 does not accurately detect the inner pressure of the fluid accommodation portion 765 if the detected level data is smaller than the first determination value level data.

For this reason, if the detected level data is smaller than the first determination value level data, the CPU 711 determines that the pressure sensor 722 has failed (S1170 in FIG. 9) and performs a predetermined failure process (S1180 in FIG. 9).

If it is determined that the pressure sensor 722 has failed, the CPU 711 outputs a predetermined alarm in the failure process (S1180 in FIG. 9). For example, the CPU 711 outputs a voice message indicating that the touch sensor 723 has failed from the speaker 790. Alternatively, the CPU 711 turns on a predetermined alarm lamp (not illustrated).

In the above-described manner, it is possible to promptly notify an operator such as a practitioner that the pressure sensor 722 has failed and thus to further improve the safety of the fluid ejection device 1.

If the detected level data of the pressure sensor 722 is equal to or greater than the first determination value level data in step S1160 of FIG. 9, the CPU 711 finishes the failure detection process.

If it is determined that the pressure sensor 722 has failed, the CPU 711 may open the pinch valve 750 in the failure process (S1100 and S1180 in FIG. 9).

In the above-described way, even if the inner pressure of the fluid accommodation portion 765 is higher than an expected pressure, the fluid in the fluid accommodation portion 765 flows via the nozzle 211 of the pulsation generator 100 and thus the inner pressure of the fluid accommodation portion 765 can be reduced. Therefore, it is possible to improve the safety of the fluid ejection device 1.

The CPU 711 may cause the fluid ejection device 1 to enter a fluid suppliable state when the pinch valve 750 is opened in the failure process (S1100 and S1180 in FIG. 9).

In the above-described way, even if the pressure sensor 722 fails, the practitioner operates the pulsation generator start-up switch 625 so as to an ON signal of the pulsation generator start-up switch 625 is input to the drive control unit 600, and thus it is possible to eject the fluid from the pulsation generator 100 in the form of a pulsed flow at a high speed.

In this case, for example, the practitioner can checks the ejection intensity by ejecting the fluid from the pulsation generator 100 as a trial, and can continuously perform an operation even if the practitioner determines that the pressure sensor 722 has failed.

There is a case where the timing at which the slider 720 comes into contact with the plunger 762 and starts pressing the plunger 762 may not be the same as the timing at which the touch sensor 723 switches from an OFF state to an ON state. Alternatively, there is a case where the timing at which the slider 720 separates from the plunger 762 may not be the same as the timing at which the touch sensor 723 switches from an ON state to an OFF state.

In this case, even if the pressure sensor 722 does not fail, a pressure detected by the pressure sensor 722 when the touch sensor 723 is in an ON state may be lower than the first determination value, or a pressure detected by the pressure sensor 722 when the touch sensor 723 is in an OFF state maybe equal to or higher than the second determination value.

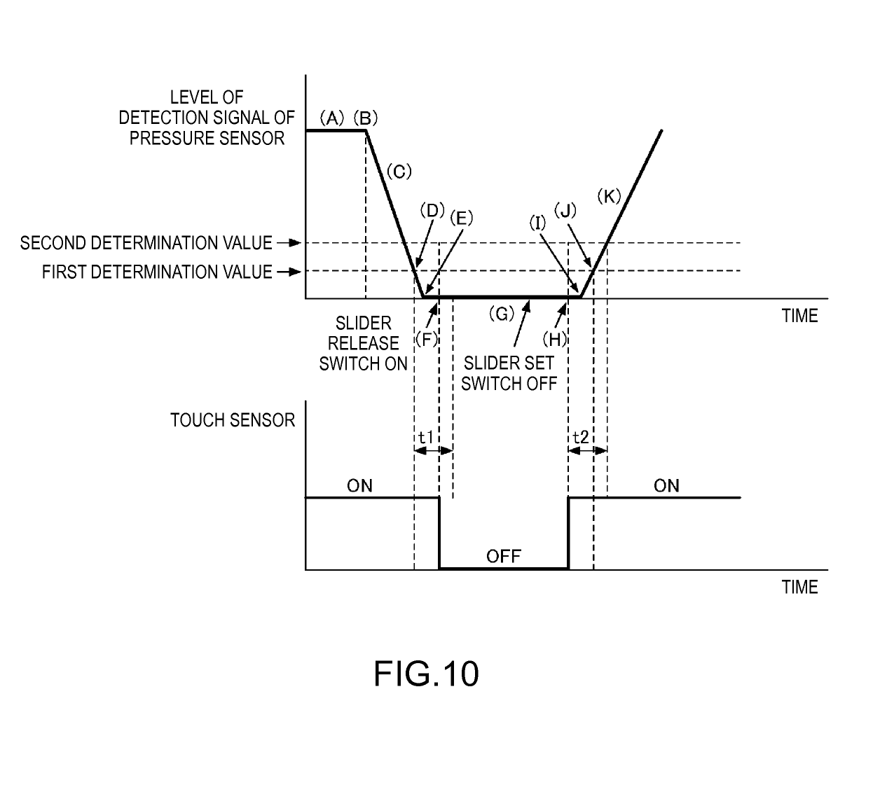

For example, in a case exemplified in FIG. 10, when the inner pressure of the fluid accommodation portion 765 is decreasing ((C) in FIG. 10), the touch sensor 723 enters an OFF state ((F) in FIG. 10) after the inner pressure of the fluid accommodation portion 765 becomes lower than the first determination value ((D) in FIG. 10). When the inner pressure of the fluid accommodation portion 765 is increasing, the inner pressure of the fluid accommodation portion 765 becomes equal to or higher than the first determination value ((J) in FIG. 10) after the touch sensor 723 enters an ON state ((H) in FIG. 10). In other words, in the case as illustrated in FIG. 10, despite the touch sensor 723 being in an ON state, there are periods in which a pressure detected by the pressure sensor 722 is lower than the first determination value ((D) to (F) and (H) to (J) in FIG. 10).

In this case, as illustrated in FIG. 10, when the inner pressure of the fluid accommodation portion 765 is decreasing, it may not be determined whether the touch sensor 723 is an ON state or an OFF state until a predetermined time t1 has elapsed after the inner pressure of the fluid accommodation portion 765 becomes lower than the first determination value ((D) in FIG. 10).

Similarly, when the inner pressure of the fluid accommodation portion 765 is increasing, it may not be determined whether or not inner pressure of the fluid accommodation portion 765 is lower than the first determination value until a predetermined time t2 has elapsed after the touch sensor 723 enters an ON state ((H) in FIGS. 10) (S1140, S1150, and S1160 in FIG. 9).

In the above-described manner, it is possible to prevent detection errors of a failure in the pressure sensor 722.

The first determination value and the second determination value may or not be the same as each other. In a case where the first determination value is different from the second determination value, it is possible to more accurately detect a failure in the pressure sensor 722 even if pressure change characteristics are different from each other during an increase and a decrease in the inner pressure of the fluid accommodation portion 765. In contrast, in a case where the first determination value is the same as the second determination value, it is possible to set simple failure determination conditions of the pressure sensor 722.

### Second Failure Detection Process

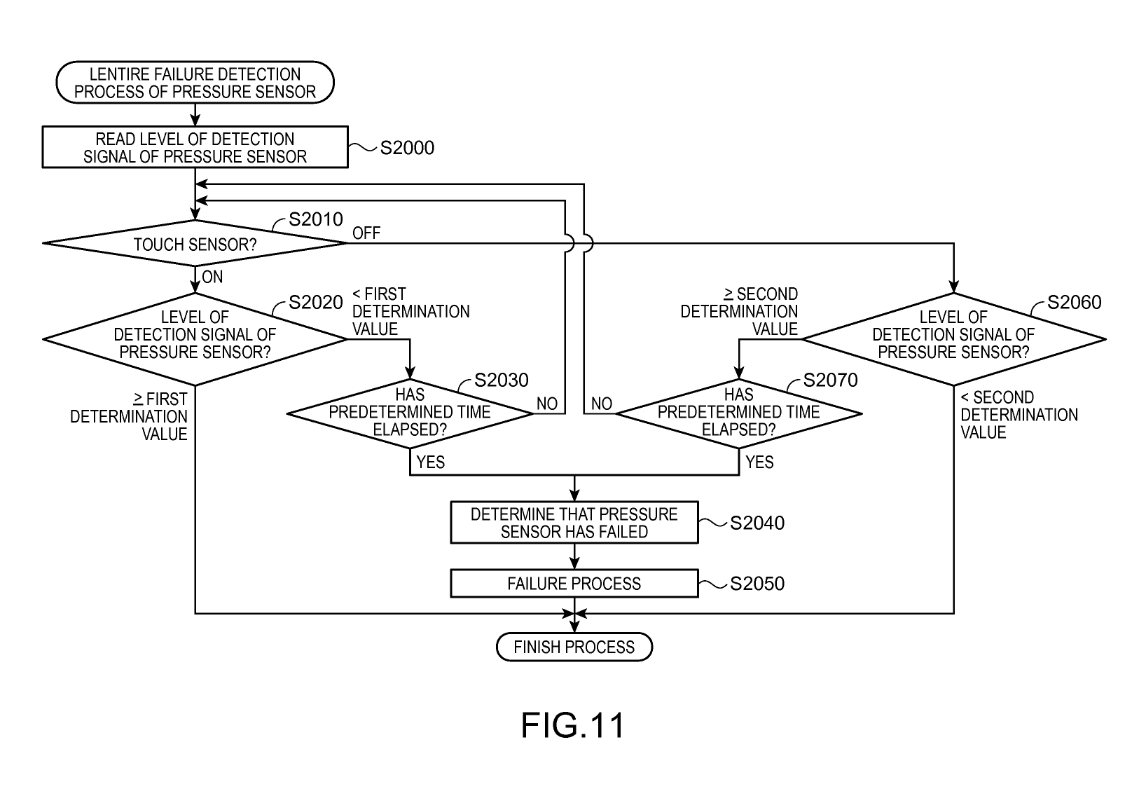

Next, a second failure detection process according to the embodiment will be described with reference to a flowchart of FIG. 11. In the second failure detection process described below, in the same manner as in the first failure detection process, the fluid ejection device 1 according to the embodiment determines that the pressure sensor 722 fails, for example, in a case where the slider (a moving body) 720 moves in the pressing direction to press the fluid accommodation portion 765 in a state in which the pinch valve (a channel opening and closing unit) 750 is closed, if a pressure detected by the pressure sensor 722 when the slider 720 comes into contact with the plunger 762 is lower than a first determination value, or if a pressure detected by the pressure sensor 722 when the slider 720 does not come into contact with the plunger 762 is equal to or higher than a second determination value; however, it is possible to detect a failure in the pressure sensor 722 even if the slider release switch 780 or the slider set switch 781 is not operated.

First, the CPU 711 acquires detected level data (a voltage value) indicating a level (a voltage) of a detection signal output from the pressure sensor 722, from the AD converter 713 (S2000 in FIG. 11).

The CPU 711 determines whether a signal output from the touch sensor 723 is an ON signal or an OFF signal (S2010 in FIG. 11).

If the output signal from the touch sensor 723 is an ON signal, the CPU 711 determines whether or not the detected level data is equal to or greater than the first determination value level data (S2020 in FIG. 11).

If the detected level data is equal to or greater than the first determination value level data, the CPU 711 finishes the failure detection process.

In contrast, if the detected level data is smaller than the first determination value level data, the CPU 711 determines whether or not the output signal from the touch sensor 723 is an ON signal, and a state in which the detected level data is smaller than the first determination value level data lasts for a first predetermined time or more (S2030 in FIG. 11).

If the state does not last for the first predetermined time or more, the CPU 711 returns to the process for determining whether the output signal from the touch sensor 723 is an ON signal or an OFF signal (S2010 in FIG. 11).

If the state lasts for the first predetermined time or more, the CPU 711 determines that the pressure sensor 722 has failed (S2040 in FIG. 11), and performs a predetermined failure process (S2050 in FIG. 11). The failure process may be the same as in the first failure detection process (S1100 and S1180 in FIG. 9).

For example, if it is determined that the pressure sensor 722 has failed, the CPU 711 outputs a predetermined alarm in the failure process (S2050 in FIG. 11). For example, the CPU 711 outputs a voice message indicating that the pressure sensor 722 has failed from the speaker 790. Alternatively, the CPU 711 turns on a predetermined alarm lamp (not illustrated).

In the above-described manner, it is possible to promptly notify an operator such as a practitioner that the pressure sensor 722 has failed and thus to further improve the safety of the fluid ejection device 1.

In contrast, if the signal output from the touch sensor 723 is an OFF signal in step S2010, the CPU 711 determines whether or not the detected level data is smaller than the second determination value level data (S2060 in FIG. 11).

If the detected level data is smaller than the second determination value level data, the CPU 711 finishes the failure detection process.

In contrast, if the detected level data is equal to or greater than the second determination value level data, the CPU 711 determines whether or not the output signal from the touch sensor 723 is an OFF signal, and a state in which the detected level data is equal to or greater than the second determination value level data lasts for a second predetermined time or more (S2070 in FIG. 11).

If the state does not last for the second predetermined time or more, the CPU 711 returns again to the process for determining whether the output signal from the touch sensor 723 is an ON signal or an OFF signal (S2010 in FIG. 11).

If the state lasts for the second predetermined time or more, the CPU 711 determines that the pressure sensor 722 has failed (S2040 in FIG. 11), and performs a predetermined failure process (S2050 in FIG. 11).

As mentioned above, in the second failure detection process, it is determined that the pressure sensor 722 has failed, if an output signal from the touch sensor 723 is an ON signal, and a state in which the detected level data is smaller than the first determination value level data lasts for the first predetermined time or more, or if an output signal from the touch sensor 723 is an OFF signal, and a state in which the detected level data is equal to or greater than the second determination value level data lasts for the second predetermined time or more.

Consequently, as exemplified in FIG. 10, even in a case where the timing at which the slider 720 comes into contact with the plunger 762 or the slider 720 separates from the plunger 762 is different from the timing at which the touch sensor 723 switches between an ON state and an OFF state, it is possible to appropriately detect a failure in the pressure sensor 722.

It is possible to detect a failure in the pressure sensor 722 without being limited to such a specific case where the slider release switch 780 or the slider set switch 781 is operated.

The first predetermined time or the second predetermined time may be set to an appropriate value through pre-tests or the like.

In the first failure detection process and the second failure detection process, the CPU 711 determines whether or not the slider 720 comes into contact with the plunger 762 by using the output signals (ON and OFF signals) from the touch sensor 723; however, there may be a form in which the output signals from the touch sensor 723 are not used.

For example, the CPU 711 may determine that the slider 720 does not come into contact with the plunger 762 when the slider 720 is located at the home position. The CPU 711 may determine that the slider 720 does not come into contact with the plunger 762 when an ON signal of the slider release switch 780 is input, the slider 720 starts moving in the opposite direction to the push-in direction, and then a predetermined time which is obtained in advance through tests or the like has elapsed.

The CPU 711 may determine that the slider 720 comes into contact with the plunger 762 when an ON signal of the slider set switch 781 is input, the slider 720 starts moving in the push-in direction, and then a predetermined time which is obtained in advance through tests or the like has elapsed.

### Other Embodiments

The fluid ejection device 1 according to the embodiment may be implemented by using the slider 720 having a plurality of pressure sensors 722.

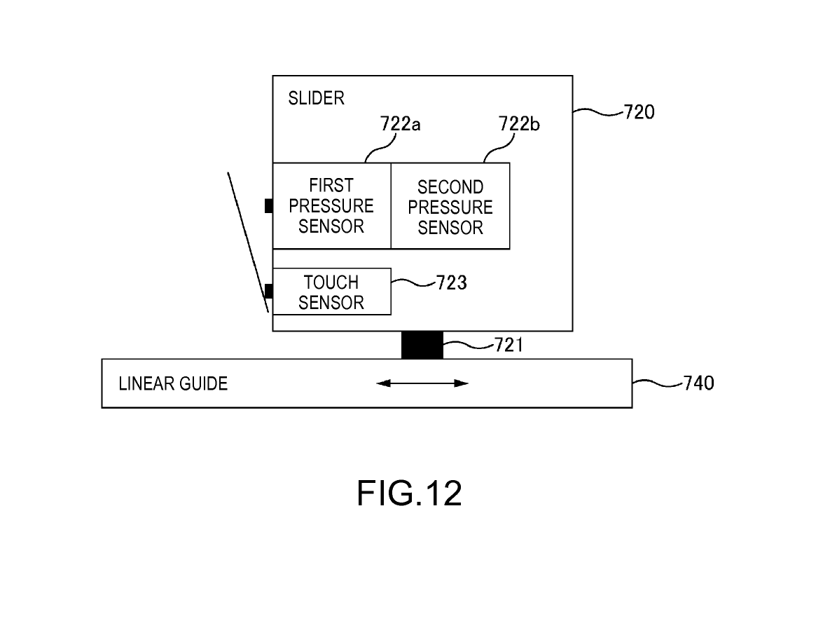

FIG. 12 is a configuration diagram in a case where the slider 720 has a first pressure sensor (a first pressure detection unit) 722a and a second pressure sensor (a second pressure detection unit) 722b.

As illustrated in FIG. 12, in the slider 720 according to the embodiment, the first pressure sensor 722a and the second pressure sensor 722b are disposed so as to be stacked in the push-in direction of the slider 720.

The first pressure sensor 722a and the second pressure sensor 722b are provided at a position where the sensors are clamped by the slider 720 and the plunger 762 when the slider 720 presses the fluid accommodation portion 765.

For this reason, a pressure when the slider 720 presses the fluid accommodation portion 765 is detected by both the first pressure sensor 722a and the second pressure sensor 722b. A first detection signal is output from the first pressure sensor 722a, and a second detection signal is output from the second pressure sensor 722b.

When receiving the first detection signal and the second detection signal, the AD converter 713 outputs first detected level data and second detected level data which are respectively correspond thereto.

The CPU 711 performs the above-described various controls on the fluid pressing unit 731 by using either one or both of the first detected level data and the second detected level data. For example, the CPU 711 moves the slider 720 so that the pressure when the slider 720 presses the fluid accommodation portion 765 becomes the predetermined target pressure value by using either one or both of the first detected level data and the second detected level data.

In a case of using both of the first detected level data and the second detected level data, the CPU 711 may calculate an average value thereof which is then used for the control. In the above-described way, the fluid pressing unit 731 can be continuously and stably controlled, for example, even in a case where either the first detection signal or the second detection signal is mixed with noise, and a value of detected level data corresponding to the signal with which the noise is mixed rapidly changes.

In a case where it is determined that the first pressure sensor 722a has failed through the above-described first failure detection process or second failure detection process, the CPU 711 continuously perform the controls by using the second pressure sensor 722b, and in a case where it is determined that the second pressure sensor 722b has failed, the CPU 711 continuously perform the controls by using the first pressure sensor 722a.

In the above-described way, the fluid ejection device 1 can continuously control ejection of the fluid from the pulsation generator 100 even if a failure has occurred in the pressure sensor 722, and thus to improve the reliability of the fluid ejection device 1.

In this case, the fluid ejection device 1 may or not output an alarm indicating that the pressure sensor 722 has failed. In the former case, the fluid ejection device 1 can continuously perform the controls by using the normal pressure sensor 722, and, in the latter case, the fluid ejection device 1 can promptly notify the operator that either one of the pressure sensors 722 has failed.

In the latter case, even if a failure has occurred in either one of the pressure sensors 722, the fluid ejection device 1 does not notify a practitioner that the failure has occurred, and thus the practitioner who concentrates on an operation is not stimulated. In this case, the occurrence of a failure in either one of the pressure sensors 722 may be preserved in the memory 712 as log information, for example. In the above-described manner, when maintenance is performed on the fluid ejection device 1 after the operation has been completed, it is possible to notify a maintenance person that the pressure sensor 722 has failed.

Alternatively, in the fluid ejection device 1 according to the embodiment, for example, in a case where a difference between the first detected level data and the second detected level data is equal to or greater than a predetermined determination value, it may be determined that at one of the first pressure sensor 722a and the second pressure sensor 722b has failed, and the above-described failure process such as outputting of a predetermined alarm may be performed.

In the above-described way, it is possible to detect a failure in the pressure sensor 722 through the first failure detection process or the second failure detection process, and also to detect a failure in the pressure sensor 722 even if the first failure detection process or the second failure detection process is not performed.

As mentioned above, although the fluid ejection device 1 according to the embodiment has been described in detail, according to the fluid ejection device 1 according to the embodiment, it is possible to implement the fluid ejection device 1 which detects a failure in the pressure sensor 722 and has higher safety and reliability.

The embodiment is presented so as to help the understanding of the invention, and does not limit the interpretation of the invention. Modifications and improvements can be made to the invention insofar as the modifications and the improvements do not depart from the spirit of the invention, and the equivalents are also included in the invention.

What is claimed is:

1. A fluid ejection device comprising:
a fluid container that has a fluid accommodation portion for accommodating a fluid, and a fluid outlet formed in the fluid accommodation portion;
a fluid pressing unit that presses the fluid accommodation portion to cause the fluid to flow out of the fluid outlet;
a connection tube, one end of which is connected to the fluid outlet;
a fluid ejection unit that has a fluid intake port connected to the other end of the connection tube, and ejects in a pulsed manner the fluid taken in via the fluid intake port;
a channel opening and closing unit that opens and closes a channel of the fluid in the connection tube;
a pressure detection unit that detects a pressure when the fluid pressing unit presses the fluid accommodation portion and outputs a detection signal with a level corresponding to the pressure; and
a control unit that causes the fluid pressing unit to press the fluid accommodation portion in a state in which the fluid opening and closing unit closes the channel, and determines that the pressure detection unit has failed in a case where the pressure indicated by the detection signal when the fluid pressing unit comes into contact with the fluid accommodation portion is lower than a first determination value, or in a case where the pressure indicated by the detection signal when the fluid pressing unit does not come into contact with the fluid accommodation portion is equal to or higher than a second determination value.

2. The fluid ejection device according to claim 1,
wherein the fluid pressing unit includes
a moving body that moves in a pressing direction of the fluid accommodation portion so as to press the fluid pressing unit; and
a position detection unit that detects a position of the moving body, and
wherein the first determination value is a value which is set on the basis of the pressure indicated by the detection signal output from the pressure detection unit when the moving body is located at a predetermined position where the moving body does not come into contact with the fluid accommodation portion.

3. The fluid ejection device according to claim 2,
wherein the first determination value is a value obtained by adding a predetermined value defined according to an error of the pressure to the pressure indicated by the detection signal output from the pressure detection unit when the moving body is located at the predetermined position.

4. The fluid ejection device according to claim 2, further comprising:
a first pressure detection unit and a second pressure detection unit,
wherein the first pressure detection unit and the second pressure detection unit are disposed so as to be stacked in the pressing direction of the moving body at a position where the pressure detection units are clamped by the moving body and the fluid accommodation unit when the moving body presses the fluid accommodation portion, and
wherein the control unit
controls the movement of the moving body so that a pressure when the moving body presses the fluid accommodation portion becomes a predetermined target pressure value by using either one or both of a first detection signal output from the first pressure detection unit and a second detection signal output from the second pressure detection unit,
continuously performs the control by using the second detection signal in a case where it is determined that the first pressure detection unit has failed, and
continuously performs the control by using the first detection signal in a case where it is determined that the second pressure detection unit has failed.

5. The fluid ejection device according to claim 1,
wherein the control unit causes the fluid opening and closing unit to open the channel in a case where it is determined that the pressure detection unit has failed.

6. The fluid ejection device according to claim 1,
wherein the control unit outputs an alarm indicating that the pressure detection unit has failed in a case where it is determined that the pressure detection unit has failed.
